(19) United States
(12) Patent Application Publication
(10) Pub. No.: US 2025/0339175 A1
(43) Pub. Date: Nov. 6, 2025

(54) PERCUTANEOUS ACCESS GUIDANCE

(71) Applicant: Auris Health, Inc., Santa Clara, CA (US)

(72) Inventors: Daniel Lau, Oakland, CA (US); Lauren Elizabeth Friend, San Francisco, CA (US); Saif Iftekar Sayed, Foster City, CA (US); Jacob William Caldwell, San Francisco, CA (US); Chiara Gatti, San Francisco, CA (US); Sean Paul Walker, Fremont, CA (US); Jialu Li, San Jose, CA (US); Namita Anil Kumar, Redwood City, CA (US)

(73) Assignee: Auris Health, Inc., Santa Clara, CA (US)

(21) Appl. No.: 19/193,102

(22) Filed: Apr. 29, 2025

Related U.S. Application Data
(60) Provisional application No. 63/641,343, filed on May 1, 2024.
(60) Provisional application No. 63/641,899, filed on May 2, 2024.
(60) Provisional application No. 63/641,909, filed on May 2, 2024.

(51) Int. Cl. A61B 17/34; A61B 17/00; A61B 34/00

(52) U.S. Cl. CPC A61B 17/3403; A61B 34/25; A61B 2017/00199; A61B 2017/00212; A61B 2034/254

(57) ABSTRACT

This disclosure provides methods, devices, and systems for percutaneous access. The present implementations more specifically relate to needle incision guidance techniques that provide real-time information about the coaxiality of a scope and a needle. In some aspects, a coaxiality indication system may generate a graphical interface that indicates a coaxiality of a needle and a scope based, at least in part, on first sensor data indicating a pose of the needle and second sensor data indicating a pose of the scope. The coaxiality of the needle and the scope may be represented by a three-dimensional model of the needle projected onto an image received from a camera disposed on the scope. Alternatively, or in addition, the coaxiality of the needle and the scope may be represented by a graphical feature depicting the orientation of the scope and orientation of the needle in relation to a common frame of reference.

### CROSS-REFERENCE TO RELATED APPLICATIONS

This Patent Application claims priority and benefit under 35 U.S.C. § 119 (e) to U.S. Provisional Patent Application No. 63/641,343, filed May 1, 2024, to U.S. Provisional Patent Application No. 63/641,899, filed May 2, 2024, and to U.S. Provisional Patent Application No. 63/641,909, filed May 2, 2024. The disclosures of all prior Applications are considered part of and are incorporated by reference in this Patent Application.

### TECHNICAL FIELD

This disclosure relates generally to medical systems, and specifically to needle incision site guidance techniques for percutaneous access.

### DESCRIPTION OF RELATED ART

Many medical procedures, such as laparoscopy, ureteroscopy, or percutaneous nephrolithotomy (PCNL), involve a series of complex steps that require careful movement and positioning of medical tools or instruments inside and/or outside a patient's body. For example, to remove urinary stones from the bladder and ureter, a medical provider (such as a physician or a technician) can insert a ureteroscope into the urinary tract through the urethra. A ureteroscope includes an endoscope at its distal end configured to enable visualization of the urinary tract. During some percutaneous access procedures, the ureteroscope can be used to designate or set a target location for a needle to access the kidney percutaneously. The medical provider inserts the needle into the patient, to the target location, and proceeds to dilate the tract and perform a PCNL procedure. For example, the medical provider may use another medical instrument (which may be in conjunction with the needle) to extract the stone from the kidney via the percutaneous access point.

In existing percutaneous access procedures, a medical provider often uses their clinical judgment in selecting a location on the surface of a patient's skin to insert the needle towards the designated target (also referred to as an “incision site”). For example, the medical provider may analyze images of the surgical field captured before and/or during the percutaneous access procedure (such as using X-ray, computed tomography (CT), and/or fluoroscopy technologies) to visualize a spatial relationship between the scope and the needle as well as the surrounding anatomy. Although various incision sites can result in a successful percutaneous access procedure, the absolute distance between the tip of the scope and the tip of the needle is minimized when the instruments are coaxially aligned (where the heading or orientation of the needle lies on the same axis as the heading or orientation of the scope). As used herein, the term “coaxiality” refers to a measure (such as an amount or degree) of coaxial alignment between a set of instruments.

The coaxiality of a needle and a scope can affect the likelihood of success of a percutaneous access procedure. However, the coaxiality of the scope and the needle can be difficult to assess from images of the surgical field (such as CT scans or X-rays). Thus, there is a need to improve upon the techniques for selecting a needle insertion site for percutaneous access.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method for guiding percutaneous access. The method includes steps of receiving first sensor data via a sensor disposed on a first instrument within an anatomy, the first sensor data indicating a pose of the first instrument; receiving an image depicting a portion of the anatomy in a field-of-view (FOV) of a camera disposed on or proximate to a distal end of the first instrument; receiving second sensor data via a sensor disposed on a second instrument external to the anatomy, the second sensor data indicating a pose of the second instrument; and generating a graphical interface that includes the image and an instrument alignment feature indicating an alignment of the second instrument with the FOV of the camera based at least in part on the first sensor data and the second sensor data.

Another innovative aspect of the subject matter of this disclosure can be implemented in a control system for guiding percutaneous access, including a processing system and a memory. The memory stores instructions that, when executed by the processing system, cause the control system to receive first sensor data via a sensor disposed on a first instrument within an anatomy, the first sensor data indicating a pose of the first instrument; receive an image depicting a portion of the anatomy in an FOV of a camera disposed on or proximate to a distal end of the first instrument; receive second sensor data via a sensor disposed on a second instrument external to the anatomy, the second sensor data indicating a pose of the second instrument; and generate a graphical interface that includes the image and an instrument alignment feature indicating an alignment of the second instrument with the FOV of the camera based at least in part on the first sensor data and the second sensor data.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows another example medical system, according to some implementations.

FIGS. 3A-3C show an example percutaneous access procedure that can be performed using the medical system of FIG. 1.

FIG. 4 shows another example medical system, according to some implementations.

FIGS. 5A and 5B show an example controller for a robotic system, according to some implementations.

FIG. 6 shows a block diagram of an example system for guiding percutaneous access, according to some implementations.

FIGS. 7A and 7B show an example operation for converting a three-dimensional (3D) instrument model in a sensor coordinate space to a two-dimensional (2D) projection in an image space.

FIG. 8 shows an example graphical interface providing instrument coaxiality guidance for incision site selection, according to some implementations.

FIG. 9 shows another example graphical interface providing instrument coaxiality guidance for incision site selection, according to some implementations.

FIG. 10 shows another example graphical interface providing instrument coaxiality guidance for incision site selection, according to some implementations.

FIG. 11 shows another example graphical interface providing instrument coaxiality guidance for incision site selection, according to some implementations.

FIG. 12 shows another example graphical interface providing instrument coaxiality guidance for incision site selection, according to some implementations.

FIGS. 13A and 13B show example images having needle alignment features overlaid thereon, according to some implementations.

FIG. 14 shows another block diagram of an example alignment indication system, according to some implementations.

FIG. 15 shows another example graphical interface providing instrument alignment guidance for incision site selection, according to some implementations.

FIG. 16 shows another example graphical interface providing instrument alignment guidance for incision site selection, according to some implementations.

FIG. 17A shows another example graphical interface providing instrument alignment guidance for incision site selection, according to some implementations.

FIG. 17B shows another example graphical interface providing instrument alignment guidance for incision site selection, according to some implementations.

FIG. 18 shows an example graphical interface providing instrument alignment guidance for needle insertion, according to some implementations.

FIG. 19 shows an example graphical interface for guided positioning of one or more robotic arms, according to some implementations.

FIG. 20 shows an example visual guide for positioning a robotic arm, according to some implementations.

FIG. 21 shows an example graphical interface for guided alignment of a sterile adapter, according to some implementations.

FIG. 22 shows an example visual guide for aligning a sterile adapter with a percutaneous access sheath, according to some implementations.

FIG. 23 shows an example graphical interface for controlling a scope, according to some implementations.

FIG. 24A shows an example graphical interface for controlling a basket retrieval device, according to some implementations.

FIG. 24B shows another example graphical interface for controlling a basket retrieval device, according to some implementations.

FIG. 25 shows an example graphical interface for controlling a catheter, according to some implementations.

FIG. 26 shows an example graphical interface for controlling a laser, according to some implementations.

FIG. 27 shows an example control system for guiding percutaneous access, according to some implementations.

FIG. 28 shows an illustrative flowchart depicting an example operation for guiding percutaneous access, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, in existing percutaneous access procedures, a medical provider (such as a physician or a technician) often uses their clinical judgment in selecting a location on the surface of a patient's skin to insert a needle (also referred to as an “incision site”) towards a target designated by a scope within an anatomy. Although various incision sites can result in a successful percutaneous access procedure, the likelihood of success is greatly increased when the instruments are coaxially aligned (where the heading or orientation of the needle lies on the same axis as the heading or orientation of the scope). As used herein, the term “coaxiality” refers to a measure (such as an amount or degree) of coaxial alignment between a set of instruments. Some medical systems implement sensing technologies (such as electromagnetic (EM) sensors) for detecting a position and an orientation (collectively referred to as a “pose”) of a needle and a scope in relation to a common coordinate system (such as an EM field). Aspects of the present disclosure recognize that such sensor data can also be used to indicate a coaxiality of the needle and the scope.

Various aspects relate generally to systems and techniques for percutaneous access, and more particularly, to needle incision guidance techniques that provide real-time information about the coaxiality of a scope and a needle. In some aspects, a coaxiality indication system may generate a graphical interface that indicates a coaxiality of a needle and a scope based, at least in part, on first sensor data received from a sensor disposed on the needle and second sensor data received from a sensor disposed on the scope. The first sensor data indicates a pose (including a position and an orientation) of the needle and the second sensor data indicates indicate a pose (including a position and an orientation) of the scope. In some implementations, the coaxiality of the needle and the scope may be represented by a graphical feature depicting the orientation of the scope and orientation of the needle in relation to a common frame of reference (such as an anterior and posterior (AP) plane and/or a cranial and caudal (CC) plane). In some other implementations, the coaxiality of the needle and the scope may be represented by a three-dimensional (3D) model of the needle projected onto an image received from a camera disposed on the scope (used for visualization of an anatomy).

Aspects of the present disclosure recognize that the field-of-view (FOV) of the camera is generally aligned with the orientation of the scope in a coordinate space associated with the sensor data (also referred to as a “sensor space”) and that the orientation of the needle in the sensor space can be depicted by the 3D model in a coordinate space associated with the camera (also referred to as a “camera space”). For example, the coaxiality indication system may align the 3D model with the orientation of the needle in the sensor space and may map the 3D model to the camera space using a calibration matrix (such as for hand-eye calibration) that maps any point or vector in the sensor space to a respective point or vector in the camera space based on the pose of the scope in the sensor space. As a result, the 3D model in the camera space can be projected onto images captured by the camera to provide real-time information about the coaxiality of the scope and the needle. For example, the coaxiality indication system may transform the 3D model into a 2D projection that depicts the relative orientation of the 3D model with respect to the orientation of the scope based on intrinsic parameters of the camera (such as an optical center, focal length, and/or skew).

Particular implementations of the subject matter described in this disclosure can be implemented to realize one or more of the following potential advantages. By generating a graphical interface depicting real-time coaxiality of a scope and a needle, aspects of the present disclosure can significantly improve the outcome of a percutaneous access operation. More specifically, the graphical interface of the present implementations can guide a medical provider to align a needle on the surface of the patient's skin to be substantially coaxial with a scope used to designate a target within an anatomy. In some implementations, the medical provider may determine that the scope and needle are coaxially aligned (such as within a threshold range of coaxiality) when the graphical interface depicts an orientation of the needle in the AP plane and/or the CC plane to be within a threshold range of an orientation of the scope in the AP plane and/or the CC plane, respectively. In some other implementations, the medical provider may determine that the scope and needle are coaxially aligned when a model of the needle displayed on the graphical interface appears to be heading (or “pointed”) in a direction substantially orthogonal to an image captured by a camera disposed on the scope.

By leveraging existing sensor data (such as EM sensor data) to generate the graphical interface, aspects of the present disclosure may further reduce the number of devices and/or workflow steps required to perform a percutaneous access procedure. For example, a medical provider may not need to use a fluoroscope or perform an intraoperative imaging operation (such as a fluoroscopy scan) to determine how and where to place the needle on the patient's skin, thereby reducing the exposure of the medical provider and the patient to harmful radiation. In addition to providing incision site guidance regarding the coaxiality of the needle and the scope, the coaxiality indication system of the present implementations also may guide the medical provider to maintain coaxial alignment between the needle and the scope throughout the needle insertion process. For example, the graphical interface may provide real-time information indicating whether a trajectory of the needle deviates from a threshold range of coaxiality as the needle is inserted towards a designated target within the anatomy.

Aspects of the present disclosure may be used to perform robotic-assisted medical procedures, such as endoscopic access, percutaneous access, or treatment for a target anatomical site. For example, robotic tools may engage or control one or more medical instruments (such as an endoscope) to access a target site within a patient's anatomy or perform a treatment at the target site. In some implementations, the robotic tools may be guided or controlled, at least in part, by a physician or technician (or other user of a medical system). In some other implementations, the robotic tools may operate in an autonomous manner. Although systems and techniques are described herein in the context of robotic-assisted medical procedures, the systems and techniques may be applicable to other types of medical procedures that utilize camera and/or sensor data (including procedures that do not rely on robotic tools or only utilize robotic tools in a very limited capacity). For example, the systems and techniques described herein may be applicable to medical procedures that rely on manually operated medical instruments (such as an endoscope that is exclusively controlled and operated by a medical provider). The systems and techniques described herein also may be applicable beyond the context of medical procedures (such as in simulated environments or laboratory settings, such as with models or simulators, among other examples).

Although certain aspects of the present disclosure are described in detail herein in the context of renal, urological, or nephrological procedures, such as kidney stone removal and treatment procedures, it should be understood that such context is provided for convenience and clarity, and the concepts disclosed herein are applicable to any suitable medical procedure (such as percutaneous endoscopic gastronomy or percutaneous endoscopic colonoscopy, among other examples). However, as mentioned, description of the renal or urinary anatomy and associated medical issues and procedures is presented herein to aid in the description of the concepts disclosed herein. In some implementations, the techniques and systems described herein are discussed in the context of a percutaneous procedure, which can include any procedure where access is gained to a target location by making a puncture or incision in the skin, mucous membrane, or other body layer. However, it should be understood that these techniques and systems can be implemented in the context of any medical procedure.

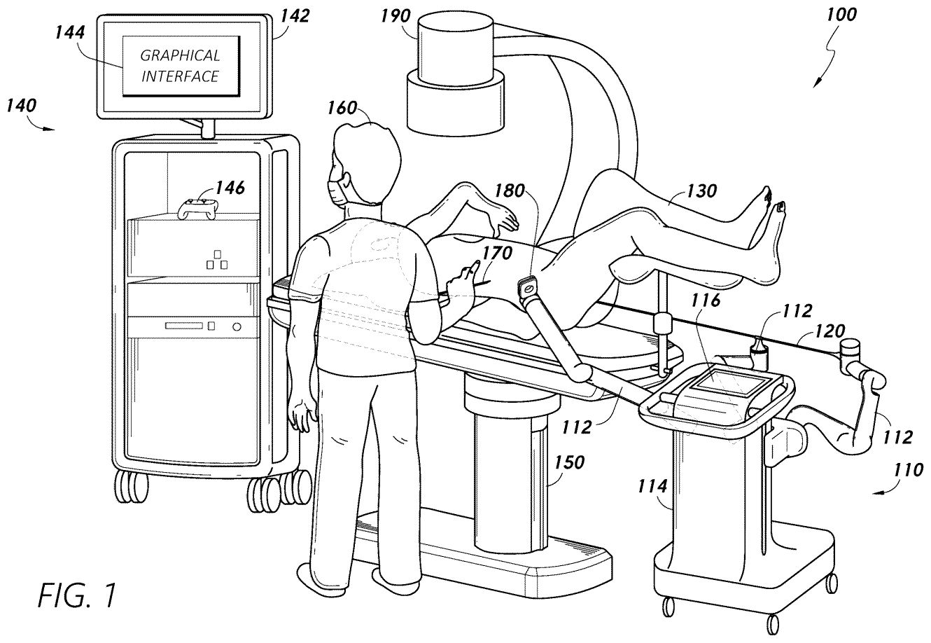

FIG. 1 shows an example medical system 100, according to some implementations. The medical system 100 includes a robotic system 110 configured to engage with and/or control a medical instrument 120 to perform a procedure on a patient 130. The medical system 100 also includes a control system 140 configured to interface with the robotic system 110, provide information regarding the procedure, and/or perform a variety of other operations. For example, the control system 140 can include one or more displays 142 to present certain information to assist the physician 160. The medical system 100 can include a table 150 configured to hold the patient 130. The system 100 further includes an electromagnetic (EM) field generator 180, which can be held by one or more robotic arms 112 of the robotic system 110 or can be a stand-alone device. In the example of FIG. 1, the medical system 100 is shown to include an imaging device 190 which can be integrated into a C-arm or otherwise configured to provide imaging during a procedure, such as for a fluoroscopy-type procedure. In some other implementations, the medical system 100 may not include the imaging device 190.

In some implementations, the medical system 100 may be used to perform a percutaneous access procedure. For example, if the patient 130 has a kidney stone that is too large to be removed through a urinary tract, the physician 160 can perform a procedure to remove the kidney stone through a percutaneous access point on the patient 130. To illustrate, the physician 160 can interact with the control system 140 to control the robotic system 110 to advance and navigate the medical instrument 120 (such as an endoscope) from the urethra, through the bladder, up the ureter, and into the kidney where the stone is located. The control system 140 can provide information via the display(s) 142 regarding the medical instrument 120 to assist the physician 160 in navigating the medical instrument 120, such as real-time images captured therewith.

Once at the site of the kidney stone (such as within a calyx of the kidney), the medical instrument 120 can be used to designate or tag a target location for the medical instrument 170 to access the kidney percutaneously (such as a desired point to access the kidney). To minimize damage to the kidney and/or the surrounding anatomy, the physician 160 can designate a particular papilla as the target location for entering into the kidney with the medical instrument 170. However, other target locations can be designated or determined. To assist the physician in inserting the medical instrument 170 into the patient 130 through the particular papilla, the control system 140 may provide an graphical interface 144, which can include a visualization to indicate an alignment of an orientation of the medical instrument 170 relative to a target trajectory (such as a desired access path), a visualization to indicate a progress of inserting the medical instrument 170 towards the target location, and/or other information. Once the medical instrument 170 has reached the target location, the physician 160 can use the medical instrument 170 and/or another medical instrument to extract the kidney stone from the patient 130, such as through the percutaneous access point.

Although the above percutaneous procedure and/or other procedures are discussed in the context of using the medical instrument 120, in some implementations a percutaneous procedure can be performed without the assistance of the medical instrument 120. Further, the medical system 100 can be used to perform a variety of other procedures. Moreover, although many implementations describe the physician 160 using the medical instrument 170, the medical instrument 170 can alternatively be used by a component of the medical system 100. For example, the medical instrument 170 can be held or manipulated by the robotic system 110 (such as the one or more robotic arms 112) and the techniques discussed herein can be implemented to control the robotic system 110 to insert the medical instrument 170 with the appropriate orientation to reach a target location.

In the example of FIG. 1, the medical instrument 120 is implemented as a scope (such as an endoscope) and the medical instrument 170 is implemented as a needle. Thus, for case of discussion, the medical instrument 120 is referred to as “the scope” or “the lumen-based medical instrument,” and the medical instrument 170 is referred to as “the needle” or “the percutaneous medical instrument.” However, the medical instrument 120 and the medical instrument 170 can each be implemented as any suitable type of medical instrument including, for example, a scope, a needle, a catheter, a guidewire, a lithotripter, a basket retrieval device, forceps, a vacuum, a needle, a scalpel, an imaging probe, jaws, scissors, graspers, needle holder, micro dissector, staple applier, tacker, suction or irrigation tool, or clip applier, among other examples. In some implementations, a medical instrument may be a steerable device. In some other implementations, a medical instrument may be a non-steerable device. A surgical tool may refer to any device that is configured to puncture or be inserted through the human anatomy, such as a needle, a scalpel, or a guidewire, among other examples. However, a surgical tool can refer to other types of medical instruments.

In some aspects, a medical instrument, such as the scope 120 and/or the needle 170, may include a sensor that is configured to generate sensor data, which can be sent to another device. In some implementations, the sensor data may indicate a pose (including a location and/or orientation) of the medical instrument and/or can be used to determine a pose of the medical instrument. For example, a sensor can include an electromagnetic (EM) sensor with a coil of conductive material. The EM field generator 180 can provide an EM field that is detected by the EM sensor on the medical instrument. The magnetic field can induce small currents in coils of the EM sensor, which can be analyzed to determine a distance and/or angle or orientation between the EM sensor and the EM field generator. In some other implementations, a medical instrument can include other types of sensors configured to generate sensor data, such as a camera, a range sensor, a radar device, a shape sensing fiber, an accelerometer, a gyroscope, an accelerometer, a satellite-based positioning sensor (such as a global positioning system (GPS)), or a radio-frequency transceiver, among other examples. In some implementations, a sensor may be positioned on a distal end of a medical instrument. In some implementations, a sensor on a medical instrument may provide sensor data to the control system 140 and the control system 140 may perform one or more localization techniques to determine or track a position and/or an orientation of the medical instrument.

The terms “scope” and “endoscope” are used herein according to their broad and ordinary meanings and can refer to any type of elongate medical instrument having image generating, viewing, and/or capturing functionality and configured to be introduced into any type of organ, cavity, lumen, chamber, and/or space of a body. For example, references herein to scopes or endoscopes can refer to a ureteroscope (such as for accessing the urinary tract), a laparoscope, a nephroscope (such as for accessing the kidneys), a bronchoscope (such as for accessing an airway, such as the bronchus), a colonoscope (such as for accessing the colon), an arthroscope (such as for accessing a joint), a cystoscope (such as for accessing the bladder), or a borescope, among other examples.

A scope can comprise a tubular and/or flexible medical instrument that is configured to be inserted into the anatomy of a patient to capture images of the anatomy. In some implementations, a scope may accommodate wires and/or optical fibers to transfer signals to or from an optical assembly and a distal end of the scope, which can include an imaging device, such as an optical camera. The camera or imaging device can be used to capture images of an internal anatomical space, such as a calyx or papilla of a kidney. A scope can further accommodate optical fibers to carry light from proximately-located light sources, such as light-emitting diodes, to the distal end of the scope. The distal end of the scope can include ports for light sources to illuminate an anatomical space when using the camera or imaging device. In some implementations, the scope may be controlled by a robotic system, such as the robotic system 110. The imaging device can comprise an optical fiber, fiber array, and/or lens. The optical components can move along with the tip of the scope such that movement of the tip of the scope results in changes to the images captured by the imaging device.

A scope can be articulable, such as with respect to at least a distal portion of the scope, so that the scope can be steered within the human anatomy. In some implementations, a scope may be articulated with, for example, five or six degrees of freedom, including X, Y, Z coordinate movement, as well as pitch, yaw, and roll. A position sensor(s) of the scope can likewise have similar degrees of freedom with respect to the position information they produce or provide. A scope can include telescoping parts, such as an inner leader portion and an outer sheath portion, which can be manipulated to telescopically extend the scope. In some aspects, a scope may comprise a rigid or flexible tube configured to be passed within an outer sheath, catheter, introducer, or other lumen-type device, or can be used without such devices. In some implementations, a scope may include a working channel for deploying medical instruments (such as lithotripters, basketing devices, or forceps), irrigation, and/or aspiration to an operative region at a distal end of the scope.

The robotic system 110 can be configured to at least partly facilitate execution of a medical procedure. The robotic system 110 can be arranged in a variety of ways depending on the particular procedure. The robotic system 110 can include the one or more robotic arms 112 configured to engage with and/or control the scope 120 to perform a procedure. As shown, each robotic arm 112 can include multiple arm segments coupled to joints, which can provide multiple degrees of movement. In the example of FIG. 1, the robotic system 110 is positioned proximate to the patient's legs and the robotic arms 112 are actuated to engage with and position the scope 120 for access into an access point, such as the urethra of the patient 130. When the robotic system 110 is properly positioned, the scope 120 can be inserted into the patient 130 robotically using the robotic arms 112, manually by the physician 160, or a combination thereof. The robotic arms 112 also can be connected to the EM field generator 180, which can be positioned near a treatment site, such as within proximity to the kidneys of the patient 130.

The robotic system 110 can include a support structure 114 coupled to the one or more robotic arms 112. The support structure 114 can include control electronics or circuitry, one or more power sources, one or more pneumatics, one or more optical sources, one or more actuators (such as motors to move the one or more robotic arms 112), memory or data storage, and/or one or more communication interfaces. In some implementations, the support structure 114 includes an input/output (I/O) device(s) 116 configured to receive input, such as user input to control the robotic system 110, and/or provide output, such as a graphical user interface (GUI), information regarding the robotic system 110, or information regarding a procedure, among other examples. The I/O device(s) 116 can include a display, a touchscreen, a touchpad, a projector, a mouse, a keyboard, a microphone, a speaker, etc. In some implementations, the robotic system 110 is movable (such as the support structure 114 includes wheels) so that the robotic system 110 can be positioned in a location that is appropriate or desired for a procedure. In other implementations, the robotic system 110 is a stationary system. Further, in some implementations, the robotic system 110 is integrated into the table 150.

The robotic system 110 can be coupled to any component of the medical system 100, such as the control system 140, the table 150, the EM field generator 180, the scope 120, and/or the needle 170. In some implementations, the robotic system is communicatively coupled to the control system 140. In one example, the robotic system 110 can be configured to receive a control signal from the control system 140 to perform an operation, such as to position a robotic arm 112 in a particular manner, or manipulate the scope 120, among other examples. In response, the robotic system 110 can control a component of the robotic system 110 to perform the operation. In another example, the robotic system 110 is configured to receive an image from the scope 120 depicting internal anatomy of the patient 130 and/or send the image to the control system 140, which can then be displayed on the display(s) 142. Furthermore, in some implementations, the robotic system 110 is coupled to a component of the medical system 100, such as the control system 140, in such a manner as to allow for fluids, optics, power, or the like to be received therefrom.

The control system 140 can be configured to provide various functionality to assist in performing a medical procedure. In some implementations, the control system 140 can be coupled to the robotic system 110 and operate in cooperation with the robotic system 110 to perform a medical procedure on the patient 130. For example, the control system 140 can communicate with the robotic system 110 via a wireless or wired connection (such as to control the robotic system 110 and/or the scope 120, receive images captured by the scope 120), provide power to the robotic system 110 via one or more electrical connections, or provide optics to the robotic system 110 via one or more optical fibers or other components, among other examples. Further, in some implementations, the control system 140 may communicate with the needle 170 and/or the scope 120 to receive sensor data from the needle 170 and/or the scope 120 (via the robotic system 110 and/or directly from the needle 170 and/or the scope 120). In some implementations, the control system 140 may communicate with the table 150 to position the table 150 in a particular orientation or otherwise control the table 150. Further, in some implementations, the control system 140 may communicate with the EM field generator 180 to control generation of an EM field around the patient 130.

The control system 140 includes various I/O devices configured to assist the physician 160 or others in performing a medical procedure. In this example, the control system 140 includes an I/O device(s) 146 that is employed by the physician 160 or other user to control the scope 120, such as to navigate the scope 120 within the patient 130. For example, the physician 160 can provide input via the I/O device(s) 146 and, in response, the control system 140 can send control signals to the robotic system 110 to manipulate the scope 120. Although the I/O device(s) 146 is illustrated as a controller in the example of FIG. 1, the I/O device(s) 146 can be implemented as a variety of types of I/O devices, such as a touchscreen, a touch pad, a mouse, or a keyboard, among other examples.

As described above, the display(s) 142 can provide a graphical interface 144 to assist the physician 160 in manipulating the needle 170. The display(s) 142 can also provide (such as via the graphical interface 144 and/or another interface) information regarding the scope 120. For example, the control system 140 can receive real-time images that are captured by the scope 120 and display the real-time images via the display(s) 142. Additionally, or alternatively, the control system 140 can receive signals (such as analog, digital, electrical, acoustic or sonic, pneumatic, tactile, or hydraulic signals) from a medical monitor and/or a sensor associated with the patient 130, and the display(s) 142 can present information regarding the health or environment of the patient 130. Such information can include information that is displayed via a medical monitor including, for example, a heart rate (such as ECG or HRV), blood pressure or rate, muscle bio-signals (such as EMG), body temperature, blood oxygen saturation (such as SpO2), CO2, brain waves (such as EEG), or environmental temperatures, among other examples.

To facilitate the functionality of the control system 140, the control system 140 can include various components or subsystems. For example, the control system 140 can include control electronics or circuitry, as well as one or more power sources, pneumatics, optical sources, actuators, memory or data storage devices, and/or communication interfaces. In some implementations, the control system 140 may include control circuitry comprising a computer-based control system that is configured to store executable instructions, that when executed, cause various operations to be implemented. In some implementations, the control system 140 may be movable (such as in FIG. 1). In some other implementations, the control system 140 may be a stationary system. Although various functionality and components are discussed as being implemented by the control system 140, any such functionality and/or components can be integrated into and/or performed by other systems and/or devices, such as the robotic system 110, the table 150, and/or the EM generator 180 (or even the scope 120 and/or the needle 170).

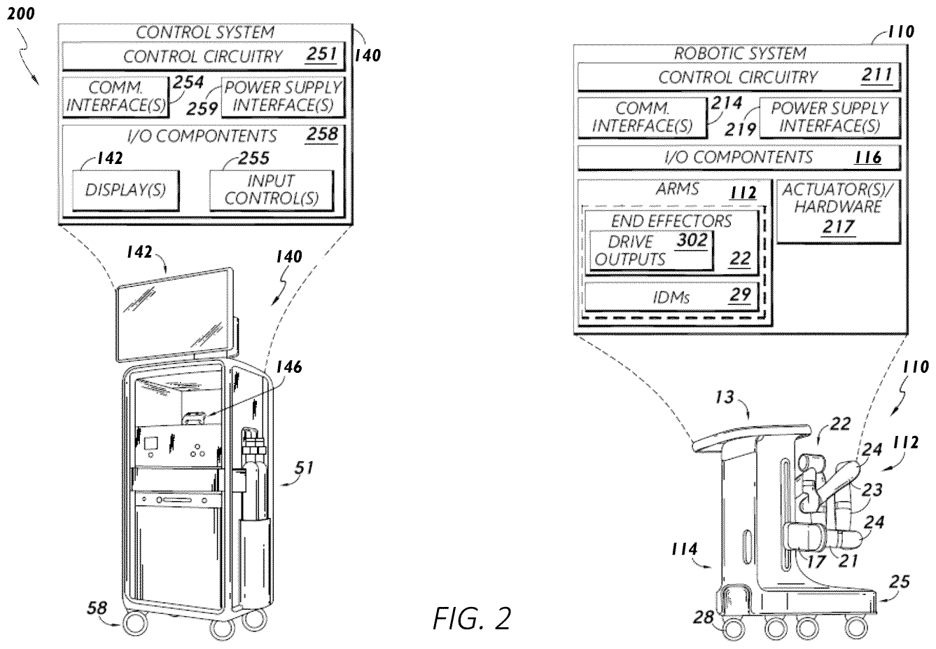

FIG. 2 shows another example medical system 200, according to some implementations. In some implementations, the medical system 200 may be one example of the medical system 100 of FIG. 1. For example, the medical system 200 is shown to include the robotic system 110 and the control system 140 of FIG. 1.

With reference to FIG. 2, the robotic system 110 includes an elongated support structure 114 (also referred to as a “column”), a robotic system base 25, and a console 13 at the top of the column 114. The column 114 may include one or more arm supports 17 (also referred to as a “carriage”) for supporting the deployment of the one or more robotic arms 112. The arm support 17 may include individually-configurable arm mounts that rotate along a perpendicular axis to adjust the base of the robotic arms 112 for better positioning relative to the patient. The robotic arms 112 may be configured to engage with and/or control the scope 120 and/or the needle 170 to perform one or more aspects of a medical procedure. For example, a scope-advancement instrument coupling (such as an instrument device manipulator) can be attached to the distal portion of one of the arms 112, to facilitate robotic control or advancement of the scope 120, while another one of the arms 112 may have associated therewith an instrument coupling that is configured to facilitate advancement of the needle 170.

The arm support 17 also includes a column interface that allows the arm support 17 to vertically translate along the column 114. In some implementations, the column interface can be connected to the column 114 through slots that are positioned on opposite sides of the column 114 to guide the vertical translation of the arm support 17. The slot contains a vertical translation interface to position and hold the arm support 17 at various vertical heights relative to the robotic system base 25. Vertical translation of the arm support 17 allows the robotic system 110 to adjust the reach of the robotic arms 112 to meet a variety of table heights, patient sizes, and physician preferences. Similarly, the individually-configurable arm mounts on the arm support 17 can allow the robotic arm base 21 of the robotic arms 112 to be angled in a variety of configurations.

The robotic arms 112 may generally comprise robotic arm bases 21 and end effectors 22, separated by a series of linkages 23 that are connected by a series of joints 24, each joint 24 comprising one or more independent actuators 217. Each actuator 217 may comprise an independently-controllable motor. Each independently-controllable joint 24 can provide an independent degree of freedom of movement to the robotic arm. In some implementations, each of the arms 112 has seven joints, and thus provides seven degrees of freedom, including “redundant” degrees of freedom. Redundant degrees of freedom allow the robotic arms 112 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The robotic system base 25 balances the weight of the column 114, arm support 17, and arms 112 over the floor. Accordingly, the robotic system base 25 may house certain relatively heavier components, such as electronics, motors, power supply, as well as components that selectively enable movement or immobilize the robotic system. For example, the robotic system base 25 can include wheel-shaped casters 28 that allow for the robotic system to easily move around the operating room prior to a procedure. After reaching the appropriate position, the casters 28 may be immobilized using wheel locks to hold the robotic system 110 in place during the procedure.

A console 13 is positioned at the upper end of column 114 and can provide one or more I/O components 116, such as a user interface for receiving user input and a display screen (or a dual-purpose device such as, for example, a touchscreen) to provide the physician or user with pre-operative and intra-operative data. Example pre-operative data may include pre-operative plans, navigation and mapping data derived from pre-operative computed tomography (CT) scans, and/or notes from pre-operative patient interviews. Example intra-operative data may include optical information provided from the tool, sensor and coordinate information from sensors, as well as vital patient statistics, such as respiration, heart rate, and/or pulse. The console 13 may be positioned and tilted to allow a physician to view the console 13, robotic arms 112, and patient while operating the console 13 from behind the robotic system 110.

The end effector 22 of each of the robotic arms 112 may comprise an instrument device manipulator (IDM) 29, which may be attached using a mechanism changer interface (MCI). In some implementations, the IDM 29 can be removed and replaced with a different type of IDM, for example, a first type of IDM may manipulate a scope, while a second type of IDM may manipulate a needle. Another type of IDM may be configured to hold an electromagnetic field generator (such as the EM field generator 180). An MCI can include connectors to transfer pneumatic pressure, electrical power, electrical signals, and/or optical signals from the robotic arm 112 to the IDM 29. The IDMs 29 may be configured to manipulate medical instruments, such as the scope 120, using techniques including, for example, direct drives, harmonic drives, geared drives, belts and pulleys, magnetic drives, and the like. In some implementations, the IDMs 29 can be attached to respective ones of the robotic arms 112, wherein the robotic arms 112 are configured to insert or retract the respective coupled medical instruments into or out of the treatment site. The robotic system 110 further includes power 219 and communication 214 interfaces (such as connectors) to transfer pneumatic pressure, electrical power, electrical signals, and/or optical signals from the robotic arms 112 to the IDMs 29.

In some implementations, a user can manually manipulate a robotic arm 112 of the robotic system 110 without using electronic user controls. For example, during setup in a surgical operating room, a user may move the robotic arms 112 and/or any other medical instruments to provide desired access to a patient. The robotic system 110 may rely on force feedback and inertia control from the user to determine appropriate configuration of the robotic arms 112 and associated instrumentation.

As described with reference to FIG. 1, the medical system 100 can include control circuitry configured to perform certain functionality described herein, including control circuitry 211 of the robotic system 110 and/or control circuitry 251 of the control system 140. That is, the control circuitry of the medical system 100 may be part of the robotic system 110, the control system 140, or some combination thereof. Therefore, any reference herein to control circuitry may refer to circuitry embodied in a robotic system, a control system, or any other component of a medical system, such as the medical system 100 shown in FIG. 1. The term “control circuitry” is used herein according to its broad and ordinary meaning, and may refer to any collection of processors, processing circuitry, processing modules or units, chips, dies (such as semiconductor dies including come or more active and/or passive devices and/or connectivity circuitry), microprocessors, micro-controllers, digital signal processors, microcomputers, central processing units, field programmable gate arrays, programmable logic devices, state machines (such as hardware state machines), logic circuitry, analog circuitry, digital circuitry, and/or any device that manipulates signals (analog and/or digital) based on hard coding of the circuitry and/or operational instructions.

Control circuitry referenced herein may further include one or more circuit substrates (such as printed circuit boards), conductive traces and vias, and/or mounting pads, connectors, and/or components. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. In implementations where control circuitry comprises a hardware and/or software state machine, analog circuitry, digital circuitry, and/or logic circuitry, data storage device(s) or register(s) storing any associated operational instructions may be embedded within, or external to, the circuitry comprising the state machine, analog circuitry, digital circuitry, and/or logic circuitry.

The control circuitry 211 and/or 251 may comprise a computer-readable medium storing, and/or configured to store, hard-coded and/or operational instructions corresponding to at least some of the steps and/or functions illustrated in one or more of the present figures and/or implementations described herein. Such computer-readable medium can be included in an article of manufacture in some instances. The control circuitry 211 and/or 251 may be locally maintained on the robotic system 110 or the control system 140 or may be remotely located at least in part (such as communicatively coupled indirectly via a local area network and/or a wide area network). Any of the control circuitry 211 and/or 251 may be configured to perform any aspect(s) of the various processes disclosed herein.

With respect to the robotic system 110, at least a portion of the control circuitry 211 may be integrated with the base 25, column 114, and/or console 13 of the robotic system 110, and/or another system communicatively coupled to the robotic system 110. With respect to the control system 140, at least a portion of the control circuitry 251 may be integrated with a console base 51 and/or display 142 of the control system 140. It should be understood that any description herein of functional control circuitry or associated functionality may be embodied in the robotic system 110, the control system 140, or any combination thereof, and/or at least in part in one or more other local or remote systems or devices.

With further reference to FIG. 2, the control system 140 can include various I/O components 258 configured to assist the physician or others in performing a medical procedure. For example, the I/O components 258 can be configured to allow for user input to control or navigate the scope 120 and/or needle 170 within the patient. In some implementations, the physician can provide input to the control system 140 and/or robotic system 110 via one or more input controls 255, wherein in response to such input, control signals can be sent to the robotic system 110 to manipulate the scope 120 and/or needle 170. Example suitable input controls 255 may include any type of user input devices or device interfaces, such as buttons, keys, joysticks, handheld controllers (such as video-game type controllers), computer mice, trackpads, trackballs, control pads, foot pedals, sensors (such as motion sensors or cameras) that capture hand or finger gestures, or touchscreens, among other examples. To facilitate the functionality of the control system 140, the control system can include various components (sometimes referred to as “subsystems”). For example, the control system 140 can include control electronics or circuitry 251, as well as one or more power supplies or supply interfaces 259, pneumatic devices, optical sources, actuators, data storage devices, and/or communication interfaces 254.

The various components of the medical system 100 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, personal area networks (PANs), body area network (BANs), etc. For example, the communication interfaces 214 and 254 of the robotic system 110 and the control system 140, respectively, can be configured to communicate with one or more devices, sensors, or systems, such as over a wireless and/or wired network connection. In some implementations, the various communication interfaces can implement a wireless technology such as Bluetooth, Wi-Fi, near field communication (NFC), or the like. Furthermore, in some implementations, the various components of the system 100 can be connected for data communication, fluid exchange, power exchange, and so on via one or more support cables, tubes, or the like.

With further reference to FIG. 1, the medical system 100 can provide a variety of benefits, such as providing guidance to assist a physician in performing a procedure (such as instrument tracking or instrument alignment information), enabling a physician to perform a procedure from an ergonomic position without the need for awkward arm motions and/or positions, enabling a single physician to perform a procedure with one or more medical instruments, avoiding radiation exposure (such as associated with fluoroscopy techniques), enabling a procedure to be performed in a single-operative setting, or providing continuous suction to remove an object more efficiently (such as to remove a kidney stone), among other examples. For example, the medical system 100 can provide guidance information to assist a physician in using various medical instruments to access a target anatomical feature while minimizing bleeding and/or damage to anatomy (such as critical organs or blood vessels).

Further, the medical system 100 can provide non-radiation based navigational and/or localization techniques and/or reduce the amount of equipment in the operating room. Moreover, the medical system 100 can provide functionality that is distributed between at least the control system 140 and the robotic system 110, which can be independently movable. Such distribution of functionality and/or mobility can enable the control system 140 and/or the robotic system 110 to be placed at locations that are optimal for a particular medical procedure, which can maximize working area around the patient and/or provide an optimized location for a physician to perform a procedure.

Although various techniques and systems are discussed as being implemented as robotically-assisted procedures (such as procedures that at least partly use the medical system 100), the techniques and systems can be implemented in other procedures, such as in fully-robotic medical procedures or human-only procedures (such as free of robotic systems). For example, the medical system 100 can be used to perform a procedure without a physician holding or manipulating a medical instrument (such as a fully-robotic procedure). That is, medical instruments that are used during a procedure, such as the scope 120 and the needle 170, can each be held or controlled by components of the medical system 100, such as the robotic arm(s) 112 of the robotic system 110.

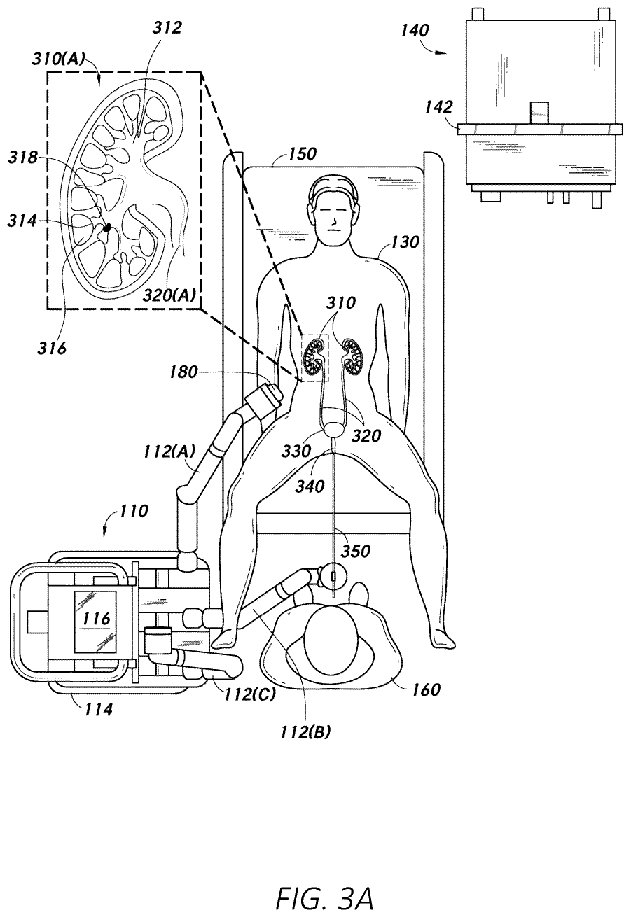

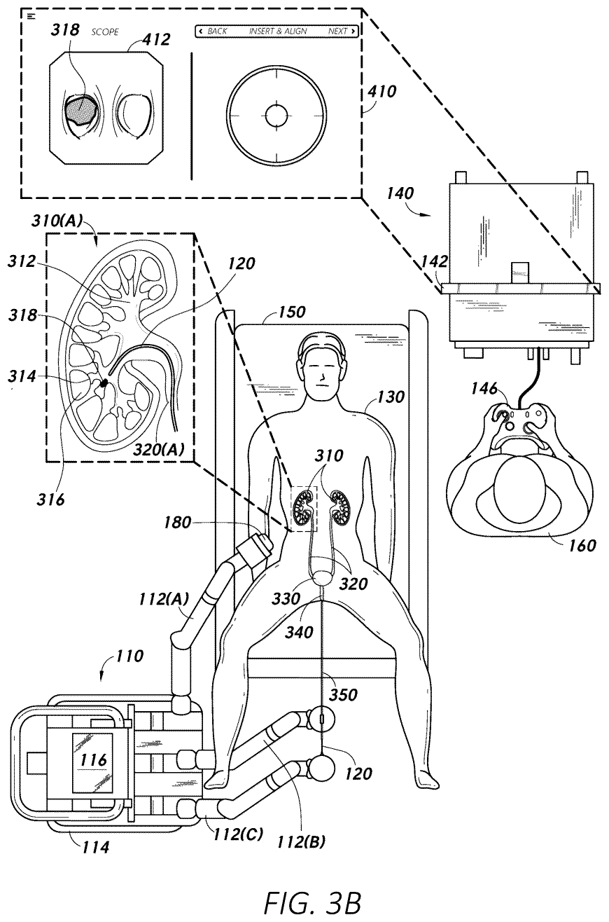

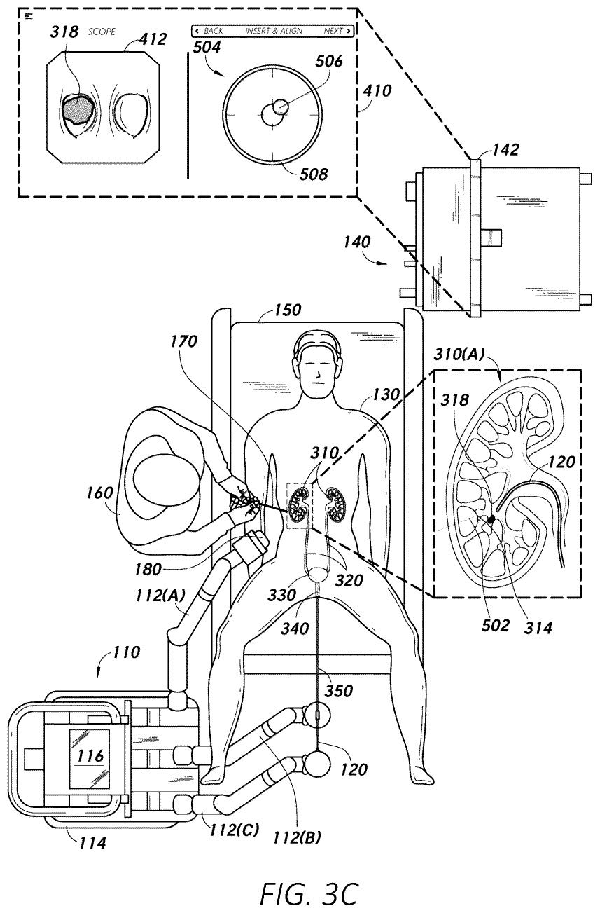

FIGS. 3A-3C show an example percutaneous access procedure that can be performed using the medical system 100 of FIG. 1. In these examples, the medical system 100 is arranged in an operating room to remove kidney stones from the patient 130 with the assistance of the scope 120 and the needle 170. In some implementations, the patient 130 may be positioned in a modified supine position with the patient 130 slightly tilted to the side to access the back or side of the patient 130, such as that illustrated in FIG. 1. However, the patient 130 can be positioned in other manners, such as a supine position or a prone position, among other examples. For case of illustration in viewing the anatomy of the patient 130, FIGS. 3A-3C illustrate the patient 130 in a supine position with the legs spread apart. Also, for case of illustration, the imaging device 190 (including the C-arm) shown in FIG. 1 has been removed.

Although FIGS. 3A-3C illustrate use of the medical system 100 to perform a percutaneous access procedure to remove a kidney stone from the patient 130, the medical system 100 can be used to remove a kidney stone in other manners and/or to perform other procedures. Further, the patient 130 can be arranged in other positions as desired for a procedure. Various acts or workflow are described with reference to FIGS. 3A-3C, and throughout this disclosure, as being performed by the physician 160. It should be understood that these acts can be performed directly by the physician 160, a user under direction of the physician 160, another user (such as a technician), a combination thereof, and/or any other user.

The renal anatomy, as illustrated at least in part in FIGS. 3A-3C, is described here for reference with respect to certain medical procedures relating to aspects of the present disclosure. The kidneys generally comprise two bean-shaped organs located on the left and right in the retroperitoneal space. The kidneys receive blood from the paired renal arteries, and blood exits into the paired renal veins. Each kidney is attached to a ureter, which is a tube that carries excreted urine from the kidney to the bladder. The bladder is attached to the urethra. A recessed area on the concave border of the kidney is the renal hilum, where the renal artery enters the kidney and the renal vein and ureter leave. The kidney is surrounded by tough fibrous tissue, the renal capsule, which is itself surrounded by perirenal fat, renal fascia, and pararenal fat. The anterior (front) surface of these tissues is the peritoneum, while the posterior (rear) surface is the transversalis fascia.

The functional substance, or parenchyma, of the kidney is divided into two major structures: the outer renal cortex and the inner renal medulla. These structures take the shape of a plurality of cone-shaped renal lobes, each containing renal cortex surrounding a portion of medulla called a renal pyramid. The tip, or papilla, of each pyramid empties urine into a respective minor calyx; minor calyces empty into major calyces, and major calyces empty into the renal pelvis, which transitions to the ureter. At the hilum, the ureter and renal vein exit the kidney and the renal artery enters. Hilar fat and lymphatic tissue with lymph nodes surrounds these structures. The hilar fat is contiguous with a fat-filled cavity called the renal sinus. The renal sinus collectively contains the renal pelvis and calyces and separates these structures from the renal medullary tissue.

FIGS. 3A-3C show various features of the anatomy of the patient 130. For example, the patient 130 includes kidneys 310 fluidly connected to a bladder 330 via ureters 320, and a urethra 340 fluidly connected to the bladder 330. As shown in the enlarged depiction of the kidney 310(A), the kidney 310(A) includes calyces (such as a calyx 312), renal papillae (such as a papilla 314), and renal pyramids (such as a pyramid 316). In these examples, a kidney stone 318 is located in proximity to the papilla 314. However, the kidney stone 318 can be located at other locations within the kidney 310(A) or elsewhere.

As shown in FIG. 3A, to remove the kidney stone 318 in the example percutaneous procedure, the physician 160 can position the robotic system 110 at the side or foot of the table 150 to initiate delivery of the scope 120 (not shown in FIG. 3A) into the patient 130. In particular, the robotic system 110 can be positioned at the side of the table 150 within proximity to the feet of the patient 130 and aligned for direct linear access to the urethra 340 of the patient 130. The hip of the patient 130 may be used as a reference point to position the robotic system 110. Once positioned, one or more of the robotic arms 112, such as the robotic arms 112(B) and 112(C), can stretch outwards to reach in between the legs of the patient 130. As shown in FIG. 3A, the robotic arm 112(B) can be controlled to extend and provide linear access to the urethra 340.

In this example, the physician 160 inserts a medical instrument 350 at least partially into the urethra 340 along this direct linear access path (also referred to as a “virtual rail”). The medical instrument 350 can include a lumen-type device configured to receive the scope 120, thereby assisting in inserting the scope 120 into the anatomy of the patient 130. By aligning the robotic arm 112(B) to the urethra 340 of the patient 130 and/or using the medical instrument 350, friction and/or forces on the sensitive anatomy in the area can be reduced. In some other implementations, the scope 120 may be inserted directly into the urethra 340 without the use of the medical instrument 350.

The physician 160 can also position the robotic arm 112(A) near a treatment site for the procedure. For example, the robotic arm 112(A) can be positioned within proximity to the incision site and/or the kidneys 310 of the patient 130. The robotic arm 112(A) can be connected to the EM field generator 180 to assist in tracking a location of the scope 120 and/or the needle 170 during the procedure. Although the robotic arm 112(A) is positioned relatively close to the patient 130, in some embodiments the robotic arm 112(A) is positioned elsewhere and/or the EM field generator 180 is integrated into the table 150 (which can allow the robotic arm 112(A) to be in a docked position). At this point in the procedure, the robotic arm 112(C) remains in a docked position, as shown in FIG. 3A. However, the robotic arm 112(C) can be used in some implementations to perform any of the functions discussed above of the robotic arms 112(A) and/or 112(C).

Once the robotic system 110 is properly positioned and/or the medical instrument 350 is inserted at least partially into the urethra 340, the scope 120 can be inserted into the patient 130 robotically, manually, or a combination thereof, as shown in FIG. 3B. For example, the physician 160 can connect the scope 120 to the robotic arm 112(C) and/or position the scope 120 at least partially within the medical instrument 350 and/or the patient 130. The scope 120 can be connected to the robotic arm 112(C) at any time, such as before the procedure or during the procedure (such as after positioning the robotic system 110). The physician 160 can then interact with the control system 140, such as the I/O device(s) 146, to navigate the scope 120 within the patient 130. For example, the physician 160 can provide input via the I/O device(s) 146 to control the robotic arm 112(C) to navigate the scope 120 through the urethra 340, the bladder 330, the ureter 320 (A), and up to the kidney 310(A).

In some aspects, the control system 140 may present an instrument-alignment interface 410 (such as the graphical interface 144 of FIG. 1) on the display(s) 142 to view a real-time image 412 captured by the scope 120 to assist the physician 160 in controlling the scope 120. The physician 160 can navigate the scope 120 to locate the kidney stone 318, as depicted in the image 412. In some implementations, the control system 140 may use localization techniques to determine a position and/or an orientation of the scope 120, which can be viewed by the physician 160 via the display(s) 142 to also assist in controlling the scope 120. Further, in some implementations, other types of information can be presented on the display(s) 142 to assist the physician 160 in controlling the scope 120, such as x-ray images of the internal anatomy of the patient 130.

Upon locating the kidney stone 318, the physician 160 can designate a target location for the needle 170 to enter the kidney 310(A) for eventual extraction of the kidney stone 318. For example, to minimize bleeding and/or avoid hitting a blood vessel or other undesirable anatomy of the kidney 310(A) and/or anatomy surrounding the kidney 310(A), the physician 160 can seek to align the needle 170 with an axis of a calyx. To do so, the physician 160 can designate a target location that is aligned with the center of the calyx and the center of a papilla (such as the papilla 314). In some implementations, the physician may designate the target by touching the scope 120 to the papilla 314 (also referred to as a “tag” position) and retracting the scope 120 to a “park” position some distance away from the papilla 314 (such as where the entire papilla 314 is within an FOV of a camera disposed on the scope 120). The control system 140 uses localization techniques to determine the “tag” and “park” positions of the scope 120 (such as based on sensor data from an EM sensor disposed on the scope 120) and sets the target location (also referred to as the “EM target”) midway between the “tag” and “park” positions.

As shown in FIG. 3C, the physician 160 can proceed with the procedure by positioning the needle 170 for insertion into the target location. In some implementations, the physician 160 may use his or her best judgment to place the needle 170 on the patient 130 at an incision site, such as based on knowledge regarding the anatomy of the patient 130, experience from previously performing the procedure, an analysis of CT or x-ray images, or other pre-operative information of the patient 130, among other examples. The physician 160 can attempt to avoid critical anatomy of the patient 130, such as the lungs, pleura, colon, paraspinal muscles, ribs, and/or intercostal nerves. In some implementations, the control system 140 may use CT, x-ray, or ultrasound images to provide information to the physician 160 regarding a location to place the needle 170 on the patient 130.

The control system 140 can determine a target trajectory 502 for inserting the needle 170 to assist the physician 160 in reaching the target location (such as the papilla 314). The target trajectory 502 can represent a desired path for accessing to the target location. The target trajectory 502 can be determined based on a position of a medical instrument (such as the needle 170 or the scope 120), a target location within the human anatomy, a position and/or orientation of a patient, or the anatomy of the patient (such as the location of organs within the patient relative to the target location), among other examples. In the example of FIG. 3C, the target trajectory 502 includes a straight line that passes through the papilla 314 and the needle 170 (extending from a tip of the needle 170 through the papilla 314, such as a point on an axis of the papilla 314).

However, the target trajectory 502 can take other forms, such as a curved line, and/or can be defined in other manners. In some implementations, the needle 170 may be a flexible bevel-tip needle that is configured to curve as the needle 170 is inserted in a straight manner. Such needle can be used to steer around particular anatomy, such as the ribs or other anatomy. Here, the control system 140 can provide information to guide a user, such as to compensate for deviation in the needle trajectory or to maintain the user on the target trajectory. Although the example of FIG. 3C illustrates the target trajectory 502 extending coaxially through the papilla 314, the target trajectory 502 can have another position, angle, and/or form. For example, a target trajectory can be implemented with a lower pole access point, such as through a papilla located below the kidney stone 318 shown in FIG. 3C, with a non-coaxial angle through the papilla, which can be used to avoid the hip.

The control system 140 can use the target trajectory 502 to provide an alignment-progress visualization 504 via the instrument-alignment interface 410. For example, the alignment-progress visualization 504 can include an instrument alignment element 506 indicative of an orientation of the needle 170 relative to the target trajectory 502. The physician 160 can view the alignment-progress visualization 504 and orient the needle 170 to the target trajectory 502. When aligned, the physician 160 can insert the needle 170 into the patient 130 to reach the target location. The alignment-progress visualization 504 may include a progress visualization 508 (also referred to as a “progress bar”) indicating a proximity of the needle 170 to the target location. Thus, the instrument-alignment interface 410 can assist the physician 160 in aligning and/or inserting the needle 170 to reach the target location.

Once the needle 170 has reached the target location, the physician 160 can insert another medical instrument (such as a power catheter, vacuum, or nephroscope) into the path created by the needle 170 and/or over the needle 170. The physician 160 can use the other medical instrument and/or the scope 120 to fragment and remove pieces of the kidney stone 318 from the kidney 310(A).

In some implementations, a position of a medical instrument can be represented with a point or point set, and an orientation of the medical instrument can be represented as an angle or offset relative to an axis or plane. For example, a position of a medical instrument can be represented with a coordinate(s) of a point or point set within a coordinate system (such as one or more X, Y, Z coordinates) and/or an orientation of the medical instrument can be represented with an angle relative to an axis or plane for the coordinate system (such as angle with respect to the X-axis or plane, Y-axis or plane, and/or Z-axis or plane). Here, a change in orientation of the medical instrument can correspond to a change in an angle of the medical instrument relative to the axis or plane. Further, in some implementations, an orientation of a medical instrument is represented with yaw, pitch, and/or roll information.

In some implementations, a trajectory may represent a pose. For example, a trajectory of a medical instrument can refer to a pose of the medical instrument, including or indicating both a position and orientation of the medical instrument. Similarly, a target trajectory can refer to a target pose, including or indicating both a position and orientation of a desired path. In some other implementations, a trajectory may refer to either an orientation or a position.

Although particular robotic arms of the robotic system 110 are illustrated as performing particular functions in the context of FIGS. 3A-3C, any of the robotic arms 112 can be used to perform the functions. Further, any additional robotic arms and/or systems can be used to perform the procedure. Moreover, the robotic system 110 can be used to perform other parts of the procedure. For example, the robotic system 110 can be controlled to align and/or insert the needle into the patient 130. To illustrate, one of the robotic arms 112 can engage with and/or control the needle 170 to position the needle 170 at the appropriate location, align the needle 170 with the target trajectory, and/or insert the needle 170 to the target location. The control system 140 can use localization techniques to perform such processing. Thus, in some implementations, a percutaneous procedure can be performed entirely or partially with the medical system 100 (such as with or without the assistance of the physician 160).

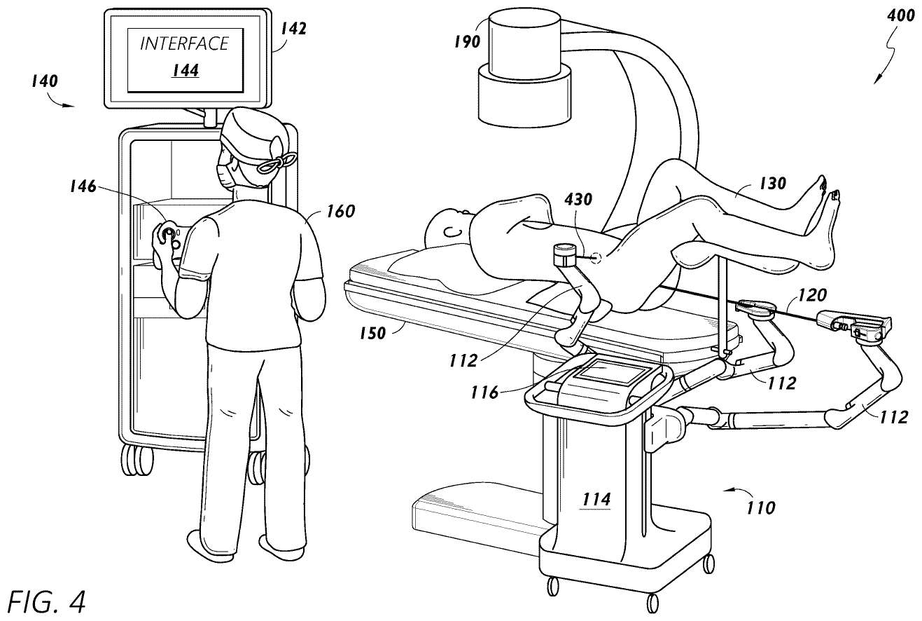

FIG. 4 shows an example medical system 400, according to some implementations. In some implementations, the medical system 400 may be one example of the medical system 100 of FIG. 1. However, the medical system 400 is shown to include a catheter 430 (instead of the needle 170) inserted percutaneously into the patient 130. For example, the catheter 430 can be inserted through an incision or opening created by the needle 170 to reach or otherwise rendezvous with a target location designated by the scope 120 (such as described with reference to FIGS. 3A-3C). In some implementations, the catheter 430 may be inserted through a sheath or shaft that has punctured the skin of the patient 130 (also referred to as a “percutaneous access sheath”).

Once the scope 120 and/or the catheter 430 are positioned at or proximate to the target location, the physician 160 can use the scope 120 to break up the kidney stone and/or use the catheter 430 to extract pieces of the kidney stone from the patient 130. For example, the scope 120 can deploy a tool (such as a laser, lithotripter, basket retrieval device, or cutting instrument) to fragment the kidney stone into pieces and the catheter 430 can suck out the pieces from the kidney through the percutaneous access path. In examples, the catheter 430 and/or the scope 120 can provide irrigation and/or aspiration to facilitate removal of the kidney stone. For instance, the catheter 430 can be coupled to an irrigation and/or aspiration system (not shown for simplicity in FIG. 4).

In some implementations, the catheter 430 may include one or more sensors configured to generate sensor data. In examples, sensor data can indicate a pose (including a position and/or orientation) of the catheter 430 and/or can be used to determine the pose of the medical instrument. Example suitable sensors can include EM sensors, cameras, range sensors, radar devices, shape sensing fibers, accelerometers, gyroscopes, accelerometers, satellite-based positioning sensors (such as GPS), and radio-frequency transceivers, among other examples. A sensor can be disposed on or coupled to a distal end of the catheter 430 and/or any other location. In some implementations, a sensor can provide sensor data to the control system 140 and/or another system/device to perform one or more localization techniques to determine/track a position and/or an orientation of the catheter 430.

A medical instrument can be associated with a coordinate frame, which can include a set of two or more vectors (or axes) that make a right angle with one another. For example, in a three-dimensional space, a coordinate frame can include three vectors (such as x-vector, y-vector, and z-vector) that make right angles with each other. Although various conventions can be used, for case of illustration the description herein will often refer to the “forward” direction (such as insert/retract) as corresponding to positive z, the “right” direction as corresponding to positive x, and the “up” direction as corresponding to positive y. The z-vector can extend along a longitudinal axis of a medical instrument. Such coordinate system can be referred to as a “left-handed coordinate system.” However, the disclosure herein can similarly be discussed/implemented in the context of a right-handed coordinate system. In examples, a coordinate frame is set or correlated based on a position of one or more elongate movement members of a medical device (such as one or more pull wires). Further, a coordinate frame can be set based on a position of an image device on a medical instrument, such as a distal end of an image device (such as a camera) on a tip of a scope. As such, a coordinate frame can correspond to a camera frame of reference. However, a coordinate frame can be set at other locations.

In the example of FIG. 4, the physician 160 can provide input via the I/O device(s) 146 and, in response, the control system 140 can send control signals to the robotic system 110 to manipulate the scope 120 and/or the catheter 430. In examples, the physician 160 can use the same I/O device to control the scope 120 and/or the catheter 430 (such as to provide user input for switching control between the devices). In some implementations, the scope 120 is driven from a first-person perspective (such as from the viewpoint of the scope 120) and the catheter 430 is driven from a third-person perspective (such as from the viewpoint of the scope 120). Although the I/O device(s) 146 is illustrated as a controller in the example of FIG. 4, the I/O device(s) 146 can be implemented as a variety of types of I/O devices, such as a touchscreen, a touch pad, a mouse, and/or a keyboard, among other examples.

In some implementations, the control system 140 can provide image data via the interface(s) 144 in a manner that maintains a constant orientation of the image data. For example, the interface(s) 144 can maintain a constant relationship with a coordinate frame for the scope 120 (so that up in the interface(s) 144 corresponds to the positive y-vector of the coordinate frame for the scope 120). To illustrate, assume that a kidney stone depicted in image data from the scope 120 initially shows up on the left side in the interface(s) 144. If the scope rolls 180 degrees, the kidney stone will move within the interface(s) 144 during the roll and appear on the right side in the interface(s) 144 after the roll. Here, the control system will not adjust the orientation of the image data displayed through the interface(s) 144. As such, the horizon in the image data can be perceived as rolling.

In other implementations, the control system 140 can provide image data via the interface(s) 144 in a manner that updates an orientation of the image data (sometimes referred to as a “rotated image or virtual view”). For example, the interface(s) 144 can update a relationship with a coordinate frame for the scope 120 (so that up in the interface(s) 144 does not always correspond to the positive y-vector of the coordinate frame for the scope 120). To illustrate, assume that a kidney stone depicted in image data from the scope 120 initially shows up on the left side in the interface(s) 144. If the scope rolls 180 degrees, the kidney stone will still show up on the left side in the interface(s) 144 after the roll. Here, the control system 140 can adjust the orientation of the image data displayed via the interface(s) 144 as the scope 120 rolls 180 degrees to maintain objects depicted in the image data in the same orientation (such as to roll correct the image data). As such, the horizon in the image data can be perceived as staying the same.

In the example of FIG. 4, two of the robotic arms 112 are actuated to engage with the scope 120 to access a target site through the urethra of the patient 130, and one of the robotic arms 112 is actuated to engage with the catheter 430 to access the target site through a percutaneous access path. When the robotic system 110 is properly positioned, the scope 120 and/or the catheter 430 can be inserted and/or navigated into the patient 130 robotically using the robotic arms 112, manually by the physician 160, or a combination thereof. Although not illustrated in FIG. 4, the robotic arms 112 can also be connected to other medical instruments, which may be interchanged during a procedure, such as an EM field generator that may be positioned near a treatment site during a particular phase of a procedure. Further, although the robotic arms 112 are shown in various positions and coupled to various instrumentation, it should be understood that such configurations are shown for convenience and illustration purposes, and such robotic arms 112 may have different configurations over time.

A control scheme can be used to map inputs to control signals to move a medical instrument. In some implementations, a control scheme includes a control frame (sometimes referred to as a “control frame of reference”), which can include an abstract coordinate frame/set of vectors that is used to control a medical instrument/device. For example, a control frame can include a set of two or more vectors (or axes) that make right angles with one another. A control frame can generally be correlated to a coordinate frame for a medical instrument. For example, a control frame for a medical instrument can be offset with respect to a coordinate frame for the medical instrument (such as 30-degree offset about an axis/vector). In examples, a coordinate frame remains static for a medical instrument (i.e., fixed to a point on the medical instrument), while a control frame can be dynamically updated, such as based on roll of the medical instrument, an orientation of image data via a user interface, and the like. In examples, a control frame is correlated to a tip of a medical instrument. However, a control frame can be correlated/centered at other locations.

In some implementations, the medical system 400 can facilitate one or more control/driving modes to assist the physician 160 in driving a medical instrument. By using multiple control modes, a medical instrument can be driven in an effective manner for different orientations of the medical instruments relative to each other. For example, if the catheter 430 is being driven from the perspective of the scope 120, the physician 160 may be able to view the catheter 430 as moving in a direction on the interface(s) 144 that more instinctively corresponds to input provided via the I/O device(s) 146. In some examples, the medical system 400 can switch to a different control mode by reconfiguring the control system 140 (such as to process an input signal from the I/O device(s) 146 and/or to generate a control signal for the robotic system 110 in a different manner), reconfiguring the I/O device(s) 146 (such as to send a different input control signal), and/or reconfiguring the robotic system 110 (such as to control a robotic arm in a different manner).

In some implementations, the control system 140 can implement a direct control mode (also referred to as a “parallel mode”) to drive a medical instrument in a corresponding manner with respect to a coordinate/control frame of the medical instrument. For example, when driving the catheter 430 from the perspective of the scope 120 in the direct control mode, if the physician 160 selects left input on the I/O device(s) 146, the control system 140 can control the catheter 430 to move left with respect to the orientation of the catheter 430. If the catheter 430 is facing in substantially the same direction as the scope 120, the physician 160 may view the catheter 430 as moving to the left in the interface(s) 144 (such as from the third-person point-of-view). In contrast, if the catheter 430 is facing the scope 120 in a head on manner, the physician 160 may view the catheter 430 as moving to the right in the interface(s) 144. Thus, the direct control mode may often be implemented when the catheter 430 and the scope 120 are substantially facing in the same direction.

Additionally, or alternatively, the control system 140 can implement an inverted control mode (also referred to as a “mirrored mode”) to drive a medical instrument in an inverted manner with respect to a coordinate/control frame of the medical instrument. For example, when driving the catheter 430 from the perspective of the scope 120 in the inverted control mode, if the physician 160 selects left input on the I/O device(s) 146, the control system 140 can control the catheter 430 to move right with respect to the orientation of the catheter 430. If the catheter 430 is facing the scope 120 in a head on manner, the physician 160 may view the catheter 430 as moving to the left in the interface(s) 144 (such as from the third-person point-of-view). In contrast, if the catheter 430 is facing in substantially the same direction as the scope 120, the physician 160 may view the catheter 430 as moving to the right in the interface(s) 144. Thus, the direct control mode may often be implemented when the catheter 430 and the scope 120 are substantially facing each other in a head on manner.

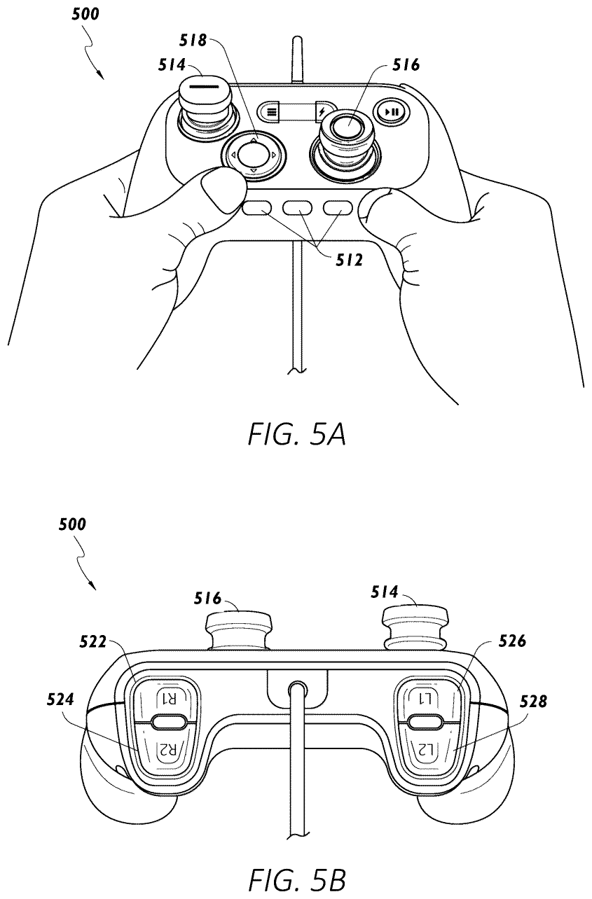

FIGS. 5A and 5B illustrate example details of a controller 500 in accordance with one or more implementations. In examples, the I/O device(s) 146 of the control system 140 and/or another I/O device discussed herein is implemented as the controller 500. However, the I/O device(s) 146 can be implemented as other types of devices. FIG. 5A and FIG. 5B illustrates a perspective view and a side view of the controller 500, respectively, according to certain implementations.

The controller 500 can receive/facilitate axis movement inputs, such via one or more joysticks 514, 516 and/or one or more directional pads 518. For example, a user can manipulate the one or more joysticks 514, 516 (and/or the one or more directional pads 518, in some cases) to provide directional input to control a medical instrument. In some implementations, the joysticks 514, 516 provide analog input while the directional pad 518 provides digital input. However, any of the joysticks 514, 516 and/or the directional pad 518 can provide analog and/or digital input. In examples, input received via the one or more directional pads 518 can be used to control a user interface, while input received via the one or more joysticks 514, 516 can be used to control movement of a medical instrument. The controller 500 can further include buttons 512 to provide additional control input. In the example illustrated in FIG. 5B, the controller 500 includes four buttons on the side of the controller: “R1” 522, “R2” 524, “L1” 526, and “L2” 528. Other implementations can include a different number of buttons and/or a different layout. In some implementations, the controller 500 can be a game-type console controller (and/or similar to a game-type console controller) repurposed to work with the control system 140. For example, controller game firmware may be overwritten with a medical device firmware and/or an input device manager can be installed in a component of the medical system 100 (such as the control system 140) to convert inputs from the controller 500 into inputs understandable by the robotic system 110.

The controller 500 can be implemented to receive input to control/drive a medical instrument. For example, the joysticks 514, 516 can receive directional input indicative of a direction to move a medical instrument (such as right, left, diagonal, up, down, insert, or retract). To illustrate, a user can tilt the joystick 516 to the left/right to cause a catheter/scope to move in a left/right direction (which can depend on a control mode) relative to a control frame, as discussed above. In another illustrate, a user can push/tilt the joystick 514 forward/back relative to FIG. 5A to cause a catheter/scope to be inserted/retracted (depending on a control mode). Although certain controls are discussed as mapping to certain functionality, the controller 500 can be configured in a variety of other manners. In some implementations, the controller 500 can be customized with a user interface that allows assigning of functionality to a particular control on the controller 500.

In some implementations, the controller 500 can implement a control (such as one or more of the controls 514-528 and/or other controls) to facilitate switching between different medical instruments. For example, a user can select one of the buttons 512 to switch from driving a scope to driving a catheter. Further, the controller 500 can implement a control to switch between control/driving modes for a medical instrument(s), such as a direct control mode, an inverted control mode, etc. Moreover, the controller 500 can implement a control to navigate to a specific interface, such as a driving interface, a calibration interface.

As described with reference to FIGS. 3A-3C, a percutaneous access procedure can be subdivided into 3 phases: a target selection phase (where a target location within an anatomy is selected or designated for percutaneous access), a site selection phase (where a needle is placed on the surface of the patient's skin and aligned with the target location), and a needle insertion phase (where the needle is driven percutaneously to rendezvous with the target location). Existing implementations of the site selection phase rely heavily (or entirely) on a physician's clinical judgment in selecting an incision site. For example, the physician may analyze three-dimensional (3D) images of the anatomy captured before and/or during the percutaneous access procedure (such as using X-ray, CT, and/or fluoroscopy technologies) to visualize a spatial relationship between the scope and the needle as well as the surrounding anatomy. However, the coaxiality of the scope and the needle can be difficult to assess from static images of the anatomy (such as CT scans or X-rays).

Although various incision sites can result in a successful percutaneous access procedure, the likelihood of success is greatly increased when the needle and the scope are coaxially aligned (where the heading or orientation of the needle lies on the same axis as the heading or orientation of the scope). As described with reference to FIGS. 1-4, some medical systems may implement sensing technologies (such as EM sensors) for detecting poses of the needle and scope in relation to a common coordinate system (such as an EM field). In some aspects, a medical system may further use such sensor data to generate a graphical interface depicting a coaxiality of the needle and the scope. In some implementations, the coaxiality of the needle and the scope may be represented by a graphical feature depicting the orientation of the scope and orientation of the needle in relation to a common frame of reference (such as an anterior and posterior (AP) plane and/or a cranial and caudal (CC) plane). In some other implementations, the coaxiality of the needle and the scope may be represented by a 3D model of the needle projected onto images received from a camera disposed on the scope.

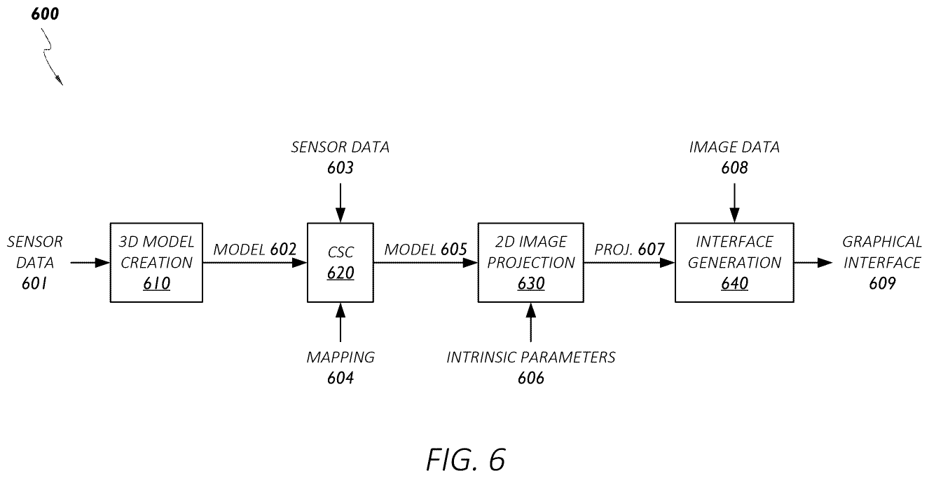

FIG. 6 shows a block diagram of an example system 600 for guiding percutaneous access, according to some implementations. In some implementations, the system 600 may be one example of any of the control circuitry 251 or 211 of FIG. 2. The system 600 is configured to produce a graphical interface 609 depicting an alignment of a needle (or other percutaneous medical instrument) relative to a scope (or other lumen-based medical instrument) based, at least in part, on sensor data 601 and 603 received via sensors disposed on the needle and the scope, respectively, and image data 608 received via a camera disposed on or proximate to the scope. In some implementations, the camera may be disposed on the distal end of the scope. In some other implementations, the camera may be disposed on the distal end of a working channel inserted through the scope (such as via a lumen of the scope). In some implementations, the sensors may be EM sensors. With reference to FIGS. 1-4, the graphical interface 609 may be one example of the graphical interface 144.

The system 600 includes a 3D model creation component 610, a coordinate space conversion (CSC) component 620, a two-dimensional (2D) image projection component 630, and an interface generation component 640. The 3D model creation component 610 is configured to produce a 3D model 602 of the needle based on the sensor data 601 received from the needle. As described with reference to FIGS. 1-4, the sensor data 601 may indicate a pose (including a position and orientation) of the needle with respect to a sensor space. For example, the sensor space may represent a world coordinate system. In some implementations, the 3D model creation component 610 may align the 3D model 602 with the pose of the needle in the sensor space. For example, the 3D model 602 may have any shape or design that reflects a general structure of a needle. Example suitable 3D models include a cone (where the base of the cone represents the circumference of the needle and the tip of the cone represents the needle tip) or a rectangular plane intersecting a circle (where the circle represents the circumference of the needle and the rectangular plane represents the needle tip).

The CSC component 620 is configured to convert the 3D model 602 from the sensor space to a corresponding 3D model 605 in a camera space associated with the camera disposed proximate to the distal end of the scope based, at least in part, on the sensor data 603 received from the scope. As described with reference to FIGS. 1-4, the sensor data 603 may indicate a pose of the scope with respect to the sensor space. The camera space represents a coordinate system that can be used to describe any point or vector in the FOV of the camera. Because the FOV of the camera is tied to the pose of the scope in the sensor space, the CSC component 620 may perform the coordinate-space conversion based on a mapping 604 that is configured to map any point or vector in the sensor space to a respective point or vector in the camera space given the position and orientation of the scope. For example, the mapping 604 may be a hand-eye calibration matrix or transform (HCAM) associated with the robotic system 110 of FIG. 1. More specifically, the hand-eye calibration matrix HEM CAM may be calibrated to estimate the pose of the scope (in the sensor space) with respect to the FOV of the camera based on a known calibration pattern.

The 2D image projection component 630 is configured to transform the 3D model 605 from the camera space to a 2D projection 607 in an image space associated with the image data 608. The image space represents a 2D slice of the camera space at a fixed distance or offset from the center of the camera (depending on the focal length of the camera). Aspects of the present disclosure recognize that any point in the 3D camera space can be projected onto the 2D image space (as depicted by the image data 608). Each projection represents a ray passing through the center of the camera and intersecting the image space at a particular point or location based on the intrinsic parameters of the camera. Example intrinsic parameters include an optical center (cₓ, c_{y}) of the camera, a focal length of the camera (fₓ, f_{y}), and a skew coefficient(s). Thus, in some implementations, the 2D image projection component 630 may transform the 3D model 605 from the camera space to the 2D projection 607 in the image space based on intrinsic parameters 606 of the camera disposed on or proximate to the distal end of the scope.

The interface generation component 640 is configured to produce the graphical interface 609 based on the image data 608 and the 2D projection 607 of the needle model. For example, the interface generation component 640 may display or render a real-time image depicting at least a portion of an anatomy in the FOV of the camera based on the image data 608. In some implementations, the interface generation component 640 may overlay the 2D projection 607 onto the image as an instrument alignment feature indicating an alignment of the needle with the FOV of the camera and/or the scope. For example, because the FOV of the camera is aligned with the orientation of the scope, the 2D projection 607 may appear to point directly at a user of the medical system (such as in a direction orthogonal to the image space) when the needle and the scope are coaxially aligned. By contrast, the 2D projection 607 may be tilted at an angle when the needle and the scope are not coaxially aligned. In some implementations, the interface generation component 640 may estimate the coaxiality of the needle and the scope based on the 2D projection 607 and display a feature on the graphical interface 609 indicating whether the instruments are coaxially aligned.

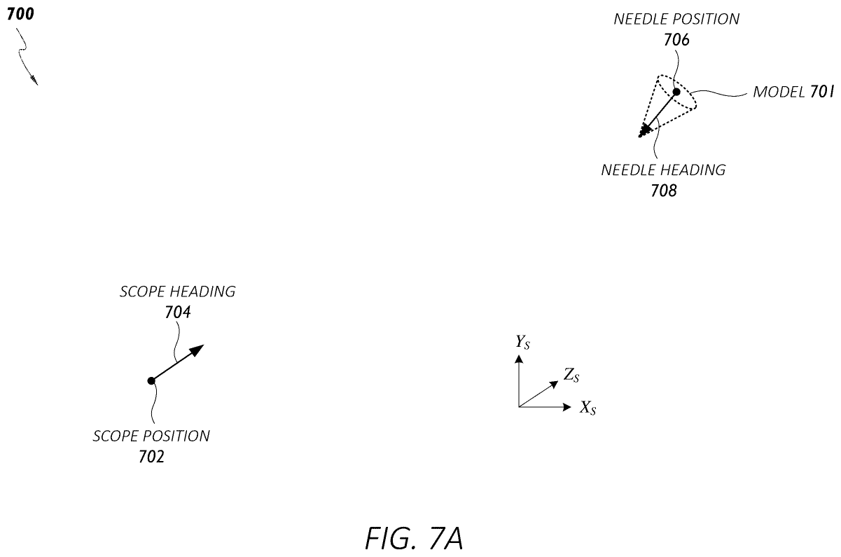

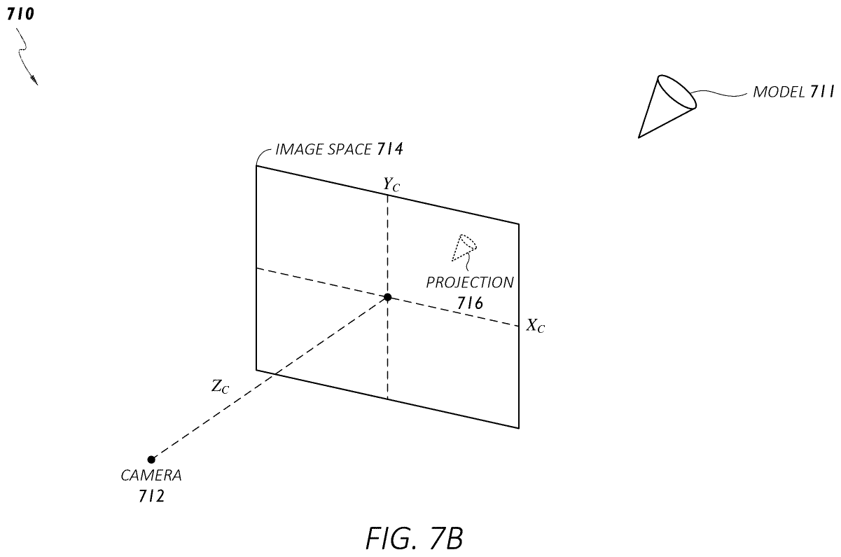

FIGS. 7A and 7B show an example operation for converting a 3D instrument model 701 in a sensor coordinate space 700 to a 2D projection 716 in an image space 714. In some implementations, the example operation may be performed by the system 600 of FIG. 6. With reference to FIG. 6, the 3D instrument model 701 may be one example of the 3D model 602 and the 2D projection 716 may be one example of the 2D projection 607.

With reference to FIG. 7A, the system 600 aligns the 3D instrument model 701 with a position 706 and a heading 708 of a needle (or other percutaneous medical instrument) in the sensor space 700 (defined by X_{S}, Y_{S}, and Z_{S} coordinates). For example, the 3D model creation component 610 may determine the needle position 706 and the needle heading 708 based on sensor data 601 received via a sensor (such as an EM sensor) disposed on the needle. The 3D model creation component 610 may further map two or more points of the 3D instrument model 701 to respective points in the sensor space 700 based on the needle position 706 and the needle heading 708. In the example of FIG. 7A, the 3D instrument model 701 is depicted as a cone having a base (which represents the circumference of the needle) centered on the needle position 706 and a tip (which represents the tip of the needle) pointing in a direction of the needle heading 708. However, the 3D instrument model 701 may have various other suitable shapes, sizes, or designs in some other implementations.

With reference to FIG. 7B, the system 600 maps the 3D instrument model 701 from the sensor space 700 to a respective 3D instrument model 711 in a camera space 710 (defined by X_{C}, Y_{C}, and Z_{C} coordinates) associated with a camera 712 disposed on or proximate to the distal end of a scope (or other lumen-based medical instrument). The camera space 710 is defined in relation to a position 702 and a heading 704 of the scope (as shown in FIG. 7A) and includes all points in the sensor space 700 lying within an FOV of the camera 712. As described with reference to FIG. 6, the CSC component 620 may convert the 3D instrument model 701 from the sensor space 700 to the camera space 710 using a hand-eye calibration matrix H_{CAM}^{EM} that transforms any point or vector in the sensor space 700 to a respective point or vector in the camera space 710 based on the scope position 702 and the scope heading 704. For example, the CSC component 620 may determine the scope position 702 and the scope heading 704 based on sensor data 603 received via a sensor (such as an EM sensor) disposed on or proximate to the distal end of the scope.

The system 600 further transforms the 3D instrument model 711 in the camera space 710 to the 2D projection 716 in the image space 714. As shown in FIG. 7B, the image space 714 represents a 2D slice (defined by the X_{C} and Y_{C} coordinates) of the 3D camera space 710. The distance from the center of the camera 712 to the image space 714 (also referred to as the “Z-offset”) depends on the focal length (fₓ, f_{y}) of the camera 712. Thus, any point in the camera space 710 can be projected onto the image space 714 via a ray passing through the center of the camera 712. As described with reference to FIG. 6, the 2D image projection component 630 may determine the 2D projection 716 of the 3D instrument model 711 based on the intrinsic parameters of the camera 712 (including its optical center (cₓ, c_{y}), focal length (fₓ, f_{y}), and skew coefficient s). For example, the intrinsic parameters can be described as a transformation matrix (K) that transforms any point or vector in the camera space 710 to a respective point or vector in the image space 714, where:

$$K = [ \begin{matrix}{f}_{x} & s & {c}_{x} \\ 0 & {f}_{y} & {c}_{y} \\ 0 & 0 & 1\end{matrix} ]$$

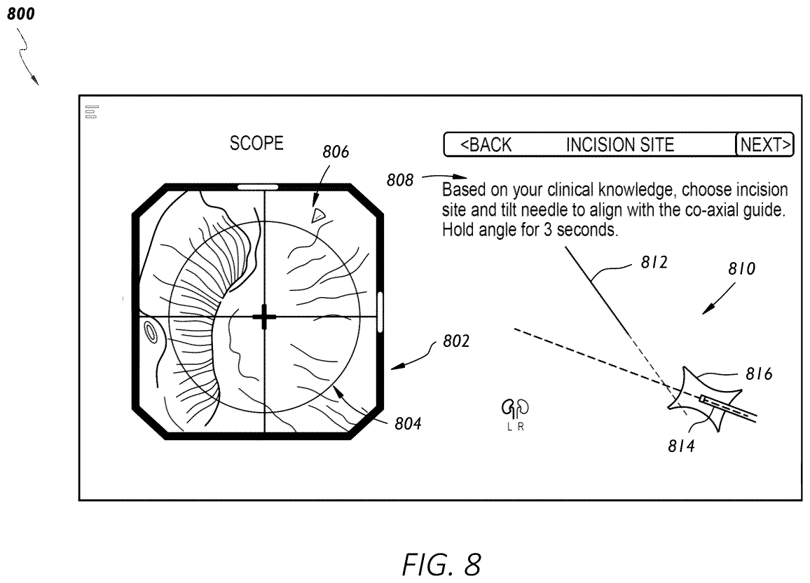

FIG. 8 shows an example graphical interface 800 providing instrument coaxiality guidance for incision site selection, according to some implementations. For example, the graphical interface 800 may be displayed to a user of a medical system (such as on the display 142 of FIG. 1) during a site selection phase of a percutaneous access procedure. In some implementations, the graphical interface 800 may be one example of the graphical interface 609 of FIG. 6. More specifically, the graphical interface 800 may help guide the user to align a needle on the surface of a patient's skin to be coaxial with a scope positioned within the patient's anatomy.

The graphical interface 800 is shown to include an image 802 depicting an FOV of a camera disposed on or proximate to the distal end of the scope and a scope alignment feature 804 overlaying the image 802. The scope alignment feature 804 is depicted as a white circle with a crosshair in the center and may be used to align the scope with a target anatomy (such as a papilla). For example, the user may position the scope so that the center of the papilla is aligned with the crosshair and the edges of the papilla are substantially aligned with the outer white circle. This may ensure that the scope is not too close or too far away from the papilla. The graphical interface 800 is also shown to include a needle alignment feature 806 overlaying the image 802. With reference to FIG. 6, the needle alignment feature 806 may be one example of the 2D projection 607. In the example of FIG. 8, the needle alignment feature 806 is depicted as a cone having a tip that points in a direction of the needle heading so that the user can assess the coaxiality of the needle and the scope. More specifically, the pose of the needle alignment feature 806 may be updated in real-time (such as based on real-time sensor data 601) to reflect any movement or tilting of the needle on the surface of the skin.

As shown in FIG. 8, the cone is tilted at an angle to indicate that the needle and the scope are not coaxially aligned. In some implementations, the graphical interface 800 may include instructions 808 to tilt the needle until it is coaxially aligned with the scope, and to hold the needle at the desired angle for a threshold duration (such as 3 seconds). The graphical interface 800 also includes a rendering 810 depicting a spatial relationship between an example needle 812 and an example scope 814. More specifically, the rendering 810 shows an example relationship between an axis of the needle 812 (depicted as a dotted line extending in a direction of heading from the tip of the needle) and an axis of the scope 814 (depicted as a dotted line extending in a direction of heading from the tip of the scope) to illustrate the concept of coaxiality. In the example of FIG. 8, the scope 814 is shown to be positioned within a calyx 816. In some implementations, the rendering 810 may be static. As such, the coaxiality of the needle 812 and the scope 814 may not reflect the actual coaxiality of the needle and the scope depicted by the needle alignment feature 806.

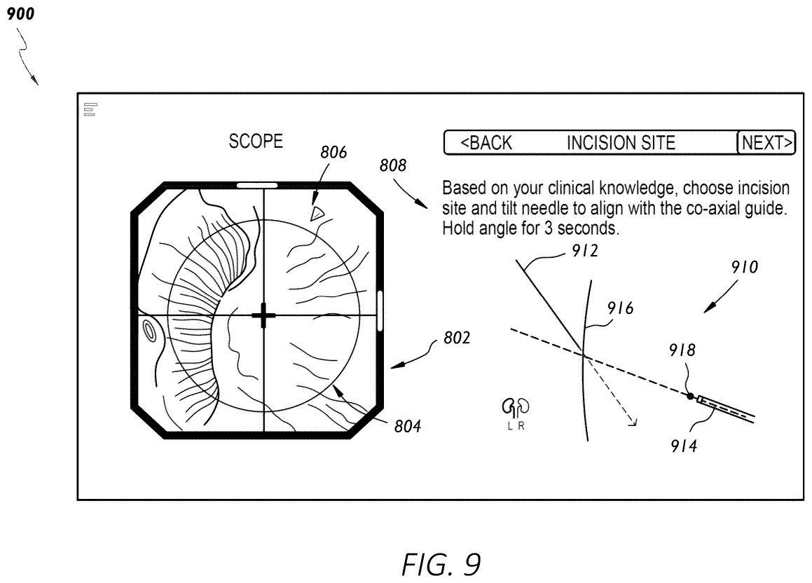

FIG. 9 shows another example graphical interface 900 providing instrument coaxiality guidance for incision site selection, according to some implementations. For example, the graphical interface 900 may be displayed to a user of a medical system (such as on the display 142 of FIG. 1) during a site selection phase of a percutaneous access procedure. In some implementations, the graphical interface 900 may be one example of the graphical interface 609 of FIG. 6. More specifically, the graphical interface 900 may help guide the user to align a needle on the surface of a patient's skin to be coaxial with a scope positioned within the patient's anatomy.

The graphical interface 900 is shown to include the image 802 of the anatomy, the scope alignment feature 804, the needle alignment feature 806, and the instructions 808 of FIG. 8. The graphical interface 900 further includes a rendering 910 depicting a spatial relationship between an example needle 912 and an example scope 914. Similar to the rendering 810 of FIG. 8, the rendering 910 shows an example relationship between an axis of the needle 912 (depicted as a dotted line extending in a direction of heading from the tip of the needle) and an axis of the scope 914 (depicted as a dotted line extending in a direction of heading from the tip of the scope) to illustrate the concept of coaxiality. In the example of FIG. 9, the needle 912 is shown to be positioned on a skin surface 916 and a designated target 918 for percutaneous access is shown in front of the scope 914. In some implementations, the rendering 910 may be static. As such, the coaxiality of the needle 912 and the scope 914 may not reflect the actual coaxiality of the needle and the scope depicted by the needle alignment feature 806.

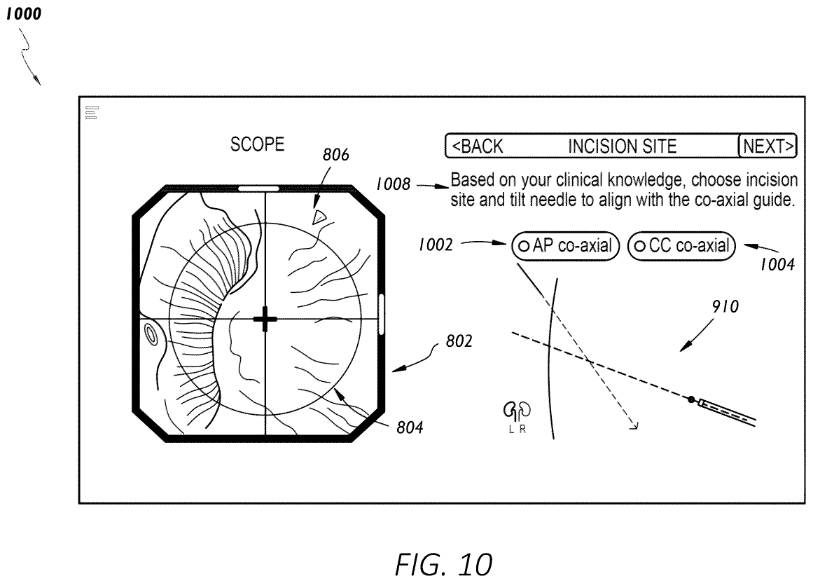

FIG. 10 shows another example graphical interface 1000 providing instrument coaxiality guidance for incision site selection, according to some implementations. For example, the graphical interface 1000 may be displayed to a user of a medical system (such as on the display 142 of FIG. 1) during a site selection phase of a percutaneous access procedure. In some implementations, the graphical interface 1000 may be one example of the graphical interface 609 of FIG. 6. More specifically, the graphical interface 1000 may help guide the user to align a needle on the surface of a patient's skin to be coaxial with a scope positioned within the patient's anatomy.

The graphical interface 1000 is shown to include the image 802 of the anatomy, the scope alignment feature 804, and the needle alignment feature 806 of FIG. 8. The graphical interface 1000 is also shown to include the rendering 910 of FIG. 9. In some implementations, the graphical interface 1000 may include instructions 1008 to tilt the needle until it is coaxially aligned with the scope. However, unlike the instructions 808 of the graphical interface 800, the instructions 1008 do not require the user to hold the needle at the desired angle for a threshold duration.

In the example of FIG. 10, the graphical interface 1000 further includes coaxial alignment indications 1002 and 1004. Each of the coaxial alignment indications 1002 and 1004 may indicate whether the needle and the scope are coaxially aligned in a respective anatomical plane. More specifically, the coaxial alignment indication 1002 may indicate whether the needle and the scope are coaxially aligned in an anterior and posterior (AP) plane, whereas the coaxial alignment indication 1004 may indicate whether the needle and the scope are coaxially aligned in a cranial and caudal (CC) plane. For example, the coaxial alignment indication 1002 may display a marking (such as a checkmark) or change colors when the needle and the scope are coaxially aligned in the AP plane. Similarly, the coaxial alignment indication 1004 may display a marking (such as a checkmark) or change colors when the needle and the scope are coaxially aligned in the CC plane.

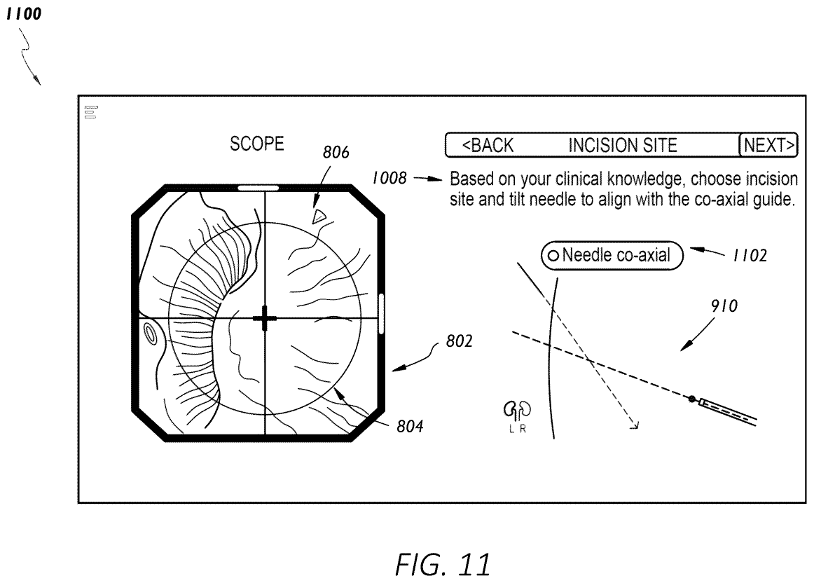

FIG. 11 shows another example graphical interface 1100 providing instrument coaxiality guidance for incision site selection, according to some implementations. For example, the graphical interface 1100 may be displayed to a user of a medical system (such as on the display 142 of FIG. 1) during a site selection phase of a percutaneous access procedure. In some implementations, the graphical interface 1100 may be one example of the graphical interface 609 of FIG. 6. More specifically, the graphical interface 1100 may help guide the user to align a needle on the surface of a patient's skin to be coaxial with a scope positioned within the patient's anatomy.

The graphical interface 1100 is shown to include the image 802 of the anatomy, the scope alignment feature 804, and the needle alignment feature 806 of FIG. 8. The graphical interface 1100 is also shown to include the rendering 910 of FIG. 9 and the instructions 1008 of FIG. 10. In the example of FIG. 11, the graphical interface 1100 further includes a coaxial alignment indication 1102. The coaxial alignment indication 1102 may indicate whether the needle and the scope are coaxially aligned. For example, the coaxial alignment indication 1102 may display a marking (such as a checkmark) or change colors when the needle and the scope are coaxially aligned. In some implementations, the coaxial alignment indication 1102 may indicate alignment if the needle and the scope are coaxially aligned in a single anatomical plane (such as the AP plane or the CC plane). In some other implementations, the coaxial alignment indication 1102 may indicate alignment only when the needle and the scope are coaxially aligned in both the AP plane and the CC plane.

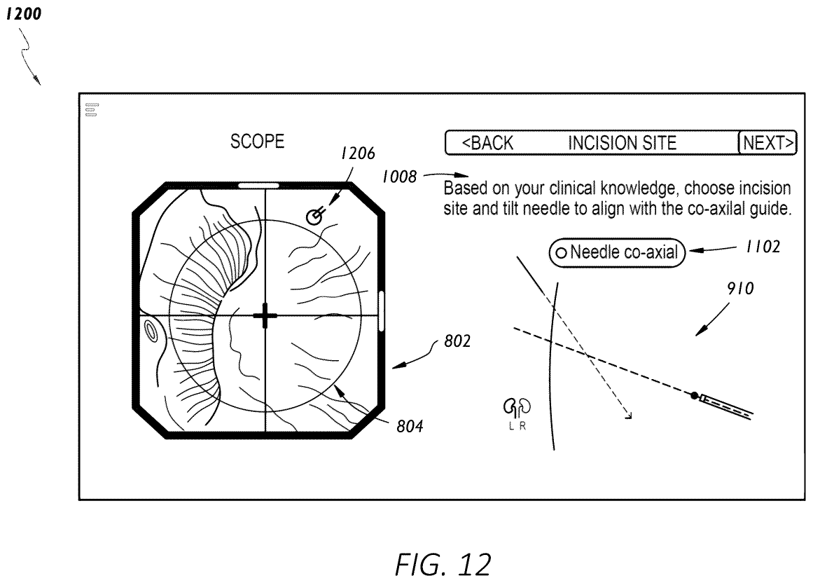

FIG. 12 shows another example graphical interface 1200 providing instrument coaxiality guidance for incision site selection, according to some implementations. For example, the graphical interface 1200 may be displayed to a user of a medical system (such as on the display 142 of FIG. 1) during a site selection phase of a percutaneous access procedure. In some implementations, the graphical interface 1200 may be one example of the graphical interface 609 of FIG. 6. More specifically, the graphical interface 1200 may help guide the user to align a needle on the surface of a patient's skin to be coaxial with a scope positioned within the patient's anatomy.

The graphical interface 1200 is shown to include the image 802 of the anatomy and the scope alignment feature 804 of FIG. 8. The graphical interface 1200 is also shown to include the rendering 910 of FIG. 9, the instructions 1008 of FIG. 10, and the coaxial alignment indication 1102 of FIG. 11. The graphical interface 1200 further includes a needle alignment feature 1206 overlaying the image 802. With reference to FIG. 6, the needle alignment feature 1206 may be one example of the 2D projection 607. In the example of FIG. 12, the needle alignment feature 1206 is depicted as a rectangular plane intersecting a circle. The rectangular plane may be deflected at an angle based on a direction of the needle heading so that the user can assess the coaxiality of the needle and the scope. Similar to the needle alignment feature 806 of FIGS. 8-11, the pose of the needle alignment feature 806 may be updated in real-time (such as based on real-time sensor data 601) to reflect any movement or tilting of the needle on the surface of the skin. As shown in FIG. 12, the rectangular plane is deflected at an angle to indicate that the needle and the scope are not coaxially aligned.

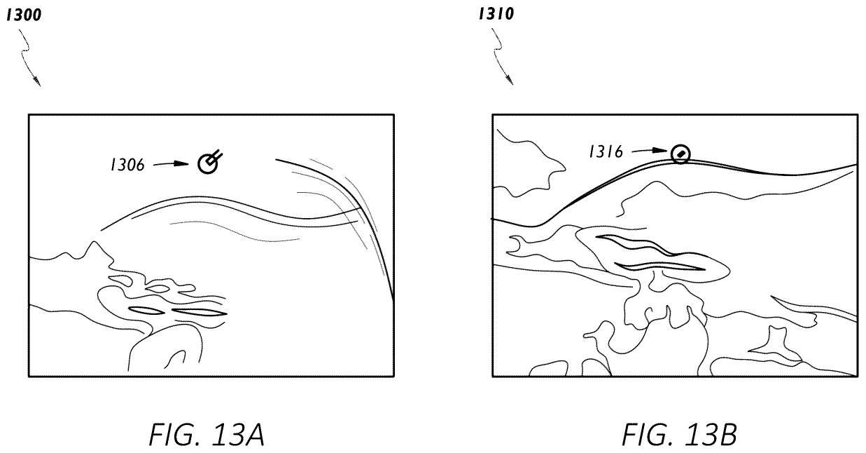

FIGS. 13A and 13B show example images 1300 and 1310, respectively, having needle alignment features 1306 and 1316 overlaid thereon, according to some implementations. In some implementations, each of the needle alignment features 1306 and 1316 may be one example of the needle alignment feature 1206 of FIG. 12. More specifically, each of the needle alignment features 1306 and 1316 is depicted as a rectangular plane intersecting a circle. With reference to FIG. 13A, the needle alignment feature 1306 may indicate that a needle and a scope are not coaxially aligned. For example, the rectangular plane is deflected at an angle to indicate that the heading of the needle is offset from the heading of the scope. With reference to FIG. 13B, the needle alignment feature 1316 may indicate that a needle and a scope are coaxially aligned. For example, the rectangular plane is orthogonal to the image space and appears as a straight line through the center of the circle.

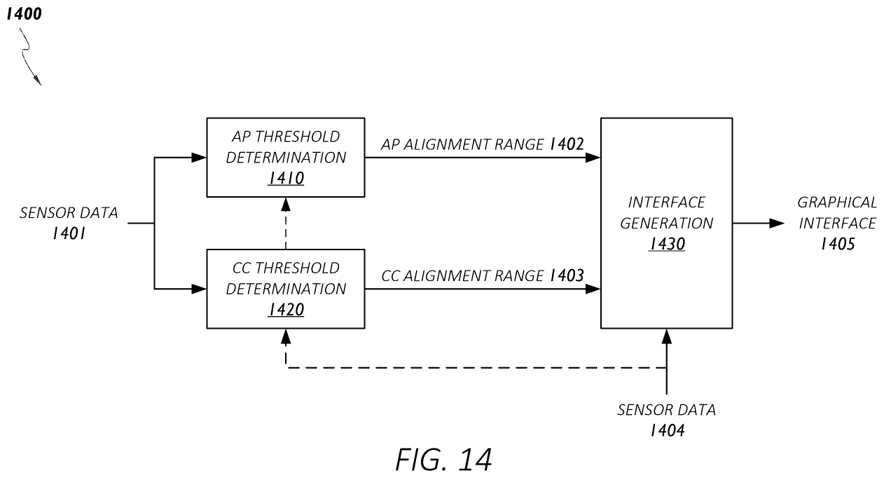

FIG. 14 shows another block diagram of an example alignment indication system 1400, according to some implementations. In some implementations, the alignment indication system 1400 may be one example of any of the control circuitry 251 or 211 of FIG. 2. The alignment indication system 1400 is configured to produce a graphical interface 1405 depicting an alignment and/or coaxiality of a needle (or other percutaneous medical instrument) and a scope (or other lumen-based medical instrument) based on sensor data 1401 and 1404 received via sensors (such as EM sensors) disposed on the needle and the scope, respectively. In some implementations, the sensors may be EM sensors. With reference to FIG. 1, the graphical interface 1405 may be one example of the graphical interface 144.

The alignment indication system 1400 includes an anterior and posterior (AP) threshold determination component 1410, a cranial and caudal (CC) threshold determination component 1420, and an interface generation component 1430. The AP threshold determination component 1410 is configured to determine a range 1402 of suitable positions and/or orientations in an AP plane for the needle to maintain a threshold degree of alignment and/or coaxiality with the scope (also referred to as an “AP alignment range”). More specifically, the AP threshold determination component 1410 may determine the AP alignment range 1402 based on a current pose of the scope, as indicated by the sensor data 1401. The CC threshold determination component 1420 is configured to determine a range 1403 of suitable positions and/or orientations in a CC plane for the needle to maintain a threshold degree of alignment and/or coaxiality with the scope (also referred to as a “CC alignment range”). More specifically, the CC threshold determination component 1410 may determine the CC alignment range 1403 based on the current pose of the scope, as indicated by the sensor data 1401.

The interface generation component 1430 is configured to produce the graphical interface 1405 based on the alignment ranges 1402 and 1403 and the sensor data 1404 received from the needle. For example, the graphical interface 1405 may depict a position and/or orientation of the needle in relation to each of the alignment ranges 1402 and 1403. The interface generation component 1430 may determine the position and/or orientation of the needle with respect to the AP plane and the CC plane based on the sensor data 1404. In some implementations, the interface generation component 1430 may display the AP alignment range 1402 along an axis of the graphical interface 1405 associated with the AP plane (also referred to as an “AP axis”) and may display the CC alignment range 1403 along an axis of the graphical interface 1405 associated with the CC plane (also referred to as a “CC axis”). In some implementations, the interface generation component 1430 may display the angle of the needle as a slider or pointer on each of the AP and CC axes. Thus, when a position and/or orientation of the needle is aligned and/or coaxial with a position and/or orientation of the scope in the AP plane, the slider on the AP axis of the graphical interface 1405 is depicted within the AP alignment range 1402. Similarly, when a position and/or orientation of the needle is aligned and/or coaxial with a position and/or orientation of the scope in the CC plane, the slider on the CC axis of the graphical interface 1405 is depicted within the CC alignment range 1403.

In some aspects, the alignment indication system 1400 may display the graphical interface 1405 during a site selection phase of a percutaneous access procedure. In such aspects, the graphical interface 1405 may guide a user of the medical system to align a position and/or orientation of the needle with a position and/or orientation of the scope on the surface of a patient's skin. In some other aspects, the alignment indication system 1400 may display the graphical interface 1405 during a needle insertion phase of a percutaneous access procedure. In such aspects, the graphical interface 1405 may guide a user of the medical system to maintain alignment between the needle and the scope as the user inserts the needle toward a designated target within a patient's anatomy. Aspects of the present disclosure recognize that the margin for error in the position and/or orientation of the needle (to achieve a successful percutaneous access) may change depending on the distance (or depth) between the needle and the scope. Thus, in some implementations, the threshold determination components 1410 and 1420 may adjust the alignment ranges 1402 and 1403, respectively, based on the insertion depth of the needle (as indicated by the sensor data 1404). For example, the alignment ranges 1402 and 1403 may be relatively large when the needle is placed on the surface of the skin and may narrow as the needle is inserted closer to the scope.

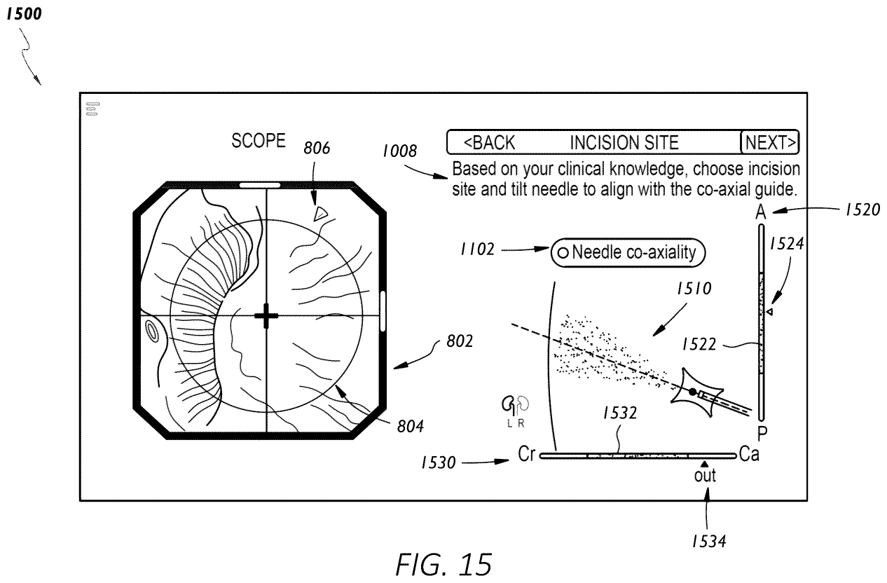

FIG. 15 shows another example graphical interface 1500 providing instrument alignment guidance for incision site selection, according to some implementations. For example, the graphical interface 1500 may be displayed to a user of a medical system (such as on the display 142 of FIG. 1) during a site selection phase of a percutaneous access procedure. In some implementations, the graphical interface 1500 may be a combination of the graphical interface 609 of FIG. 6 and the graphical interface 1405 of FIG. 14. More specifically, the graphical interface 1500 may help guide the user to align a position and orientation of a needle on the surface of a patient's skin with a position and orientation of a scope to achieve the greatest likelihood of a successful percutaneous access.

The graphical interface 1500 is shown to include the image 802 of the anatomy, the scope alignment feature 804, and the needle alignment feature 806 of FIG. 8. The graphical interface 1500 is also shown to include the instructions 1008 of FIG. 10 and the coaxial alignment indication 1102 of FIG. 11. The graphical interface 1500 further depicts an AP axis 1520 (which represents an anterior (A) and posterior (P) plane) and a CC axis 1530 (which represents a cranial (Cr) and caudal (Ca) plane) having “lanes” 1522 and 1532, respectively, overlaid thereon. In some implementations, the lanes 1522 and 1532 may be examples of the alignment ranges 1402 and 1403, respectively, of FIG. 14. More specifically, the height of the vertical lane 1522 depicts a range of suitable positions and/or orientations for the needle to maintain a threshold degree of alignment with the current position and orientation of the scope in the AP plane. Similarly, the width of the horizontal lane 1532 depicts a range of suitable positions and/or orientations for the needle to maintain a threshold degree of alignment with the current position and orientation of the scope in the CC plane. The graphical interface 1500 also includes sliders 1524 and 1534 indicating the current position and/or orientation of the needle with respect to the axes 1520 and 1530, respectively.

As shown in FIG. 15, the slider 1524 is within the vertical lane 1522, which indicates that the positions and/or orientations of the needle and the scope are aligned (at least to a threshold degree) in the AP plane. By contrast, the slider 1534 is outside the horizontal lane 1532, which indicates that the positions and/or orientations of the needle and the scope are not aligned in the CC plane. In some implementations, the slider 1534 may include additional coloring and/or text to further indicate that the needle and the scope are not aligned in the CC plane. The graphical interface 1500 further includes a rendering 1510 depicting a spatial relationship between the axis or heading of the scope and a range of suitable trajectories for the needle represented by the lanes 1522 and 1532. As shown in FIG. 15, the lanes 1522 and 1532 describe a range of positions and/or orientations, relative to the axis of the scope, centered around a designated target in front of the scope (such as in the shape of a cone). In the example of FIG. 15, the graphical interface 1500 is shown to include multiple alignment indications (such as the needle alignment feature 806, the lanes 1522 and 1532, and the sliders 1524 and 1532). However, in actual implementations, the graphical interface 1500 may include only a subset of the alignment indications depicted in FIG. 15.

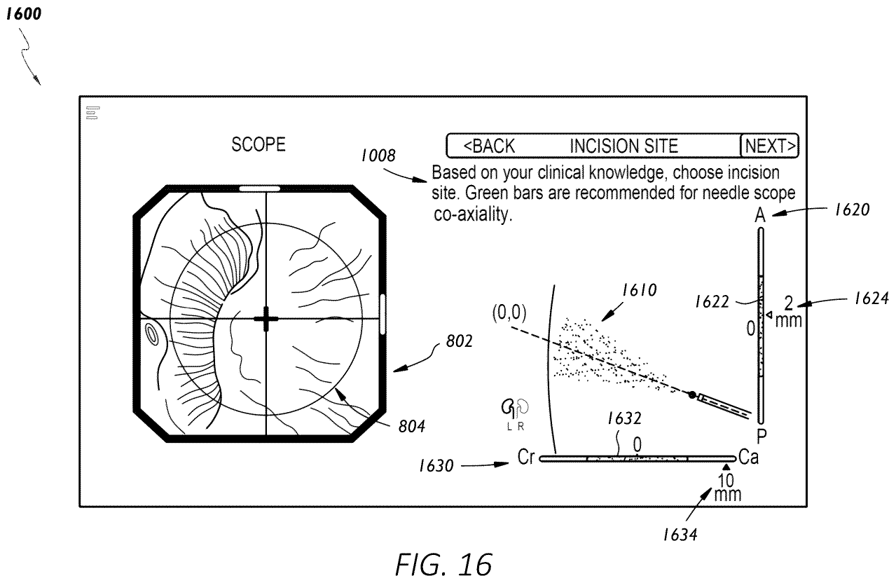

FIG. 16 shows another example graphical interface 1600 providing instrument alignment guidance for incision site selection, according to some implementations. For example, the graphical interface 1600 may be displayed to a user of a medical system (such as on the display 142 of FIG. 1) during a site selection phase of a percutaneous access procedure. In some implementations, the graphical interface 1600 may be one example of the graphical interface 1405 of FIG. 14. More specifically, the graphical interface 1600 may help guide the user to align a position of a needle on the surface of a patient's skin with a position and orientation of a scope to achieve the greatest likelihood of a successful percutaneous access.

The graphical interface 1600 is shown to include the image 802 of the anatomy and the scope alignment feature 804 of FIG. 8. The graphical interface 1600 is also shown to include the instructions 1008 of FIG. 10. The graphical interface 1600 further depicts an AP axis 1620 (which represents an anterior (A) and posterior (P) plane) and a CC axis 1630 (which represents a cranial (Cr) and caudal (Ca) plane) having “lanes” 1622 and 1632, respectively, overlaid thereon. In some implementations, the lanes 1622 and 1632 may be examples of the alignment ranges 1402 and 1403, respectively, of FIG. 14. More specifically, the height of the vertical lane 1622 depicts a range of suitable positions for the needle to maintain a threshold degree of alignment with the current position and orientation of the scope in the AP plane. Similarly, the width of the horizontal lane 1632 depicts a range of suitable positions for the needle to maintain a threshold degree of alignment with the current position and orientation of the scope in the CC plane. The graphical interface 1600 also includes sliders 1624 and 1634 indicating the current position and of the needle with respect to the axes 1620 and 1630, respectively.

As shown in FIG. 16, the slider 1624 is within the vertical lane 1622 (at a distance of 2 mm away from the scope's axis of heading), which indicates that the position of the needle is aligned with the position and orientation of the scope (at least to a threshold degree) in the AP plane. By contrast, the slider 1634 is outside the horizontal lane 1632 (at a distance of 10 mm away from the scope's axis of heading), which indicates that the position of the needle is not aligned with the position and orientation of the scope in the CC plane. In some implementations, the slider 1634 may include additional coloring and/or text to further indicate that the needle and the scope are not aligned in the CC plane. The graphical interface 1600 further includes a rendering 1610 depicting a spatial relationship between the axis or heading of the scope (0,0) and a range of suitable positions for the needle represented by the lanes 1622 and 1632. As shown in FIG. 16, the lanes 1622 and 1632 describe a range of distances, relative to the axis of the scope (0,0), centered around a designated target in front of the scope (such as in the shape of a cone).

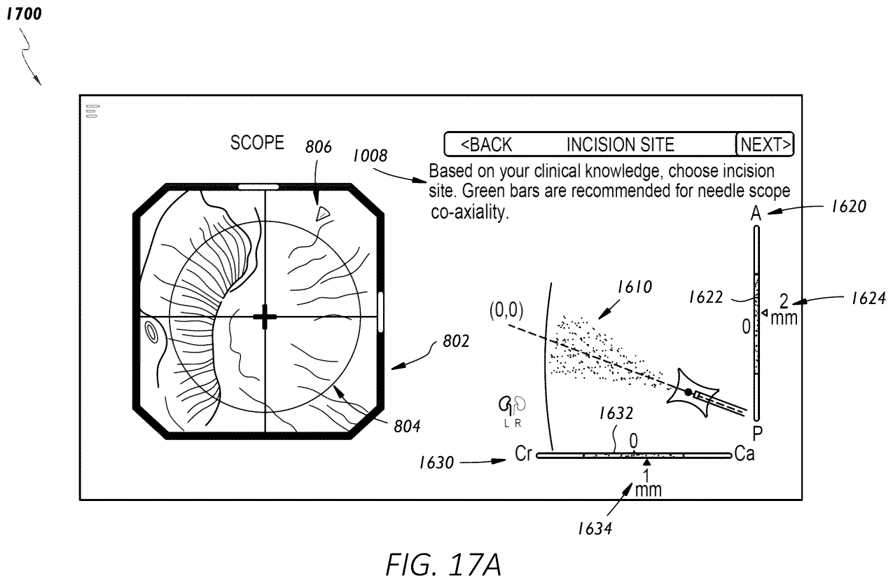

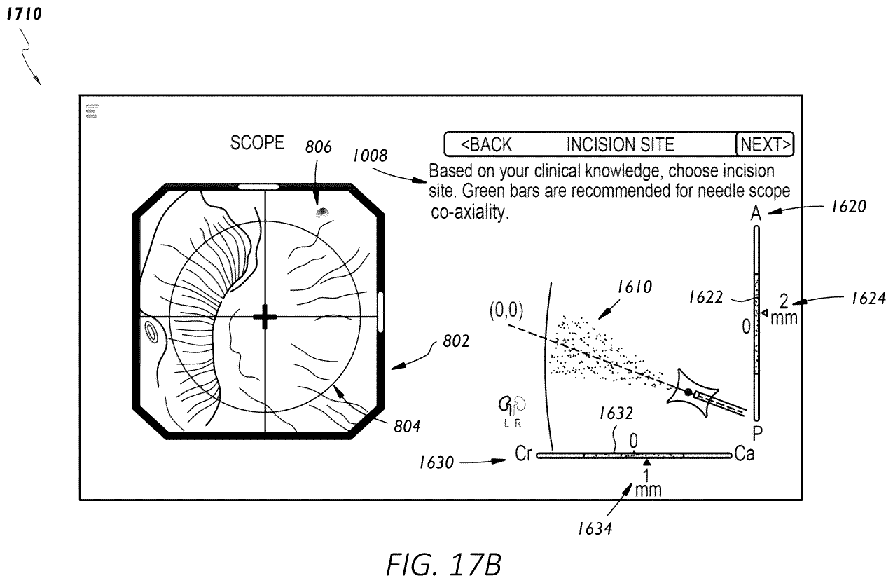

FIG. 17A shows another example graphical interface 1700 providing instrument alignment guidance for incision site selection, according to some implementations. For example, the graphical interface 1700 may be displayed to a user of a medical system (such as on the display 142 of FIG. 1) during a site selection phase of a percutaneous access procedure. In some implementations, the graphical interface 1700 may be a combination of the graphical interface 609 of FIG. 6 and the graphical interface 1405 of FIG. 14. More specifically, the graphical interface 1700 may help guide the user to align a position and orientation of a needle on the surface of a patient's skin with a position and orientation of a scope to achieve the greatest likelihood of a successful percutaneous access.

The graphical interface 1700 is shown to include the image 802 of the anatomy, the scope alignment feature 804, and the needle alignment feature 806 of FIG. 8, as well as the instructions 1008 of FIG. 10. With reference to FIG. 16, the graphical interface 1700 further includes the lanes 1622 and 1632 and the sliders 1624 and 1634, in relation to the axes 1620 and 1630, as well as the rendering 1610 depicting a spatial relationship between the axis or heading of the scope (0,0) and a range of suitable positions for the needle represented by the lanes 1622 and 1632. In the example of FIG. 17A, the graphical interface 1700 is shown to include multiple alignment indications (such as the needle alignment feature 806, the lanes 1622 and 1632, and the sliders 1624 and 1632). However, in actual implementations, the graphical interface 1700 may include only a subset of the alignment indications depicted in FIG. 17A.

As shown in FIG. 17A, the slider 1624 is within the vertical lane 1622 (at a distance of 2 mm away from the scope's axis of heading), which indicates that the position of the needle is aligned with the position and orientation of the scope (at least to a threshold degree) in the AP plane. Similarly, the slider 1634 is within the horizontal lane 1632, which indicates that the position of the needle is aligned with the position and orientation of the scope (at least to a threshold degree) in the CC plane. However, the needle alignment feature 806 indicates that the needle and the scope are not coaxially aligned (as indicated by the tip of the cone pointing too far in the posterior direction). Thus, the user may need to adjust the orientation of the needle before proceeding further with the current percutaneous access procedure.

FIG. 17B shows another example graphical interface 1710 providing instrument alignment guidance for incision site selection, according to some implementations. For example, the graphical interface 1710 may be displayed to a user of a medical system (such as on the display 142 of FIG. 1) during a site selection phase of a percutaneous access procedure. In some implementations, the graphical interface 1710 may be a combination of the graphical interface 609 of FIG. 6 and the graphical interface 1405 of FIG. 14. More specifically, the graphical interface 1710 may help guide the user to align a position and orientation of a needle on the surface of a patient's skin with a position and orientation of a scope to achieve the greatest likelihood of a successful percutaneous access.

The graphical interface 1710 is shown to include the image 802 of the anatomy, the scope alignment feature 804, and the needle alignment feature 806 of FIG. 8, as well as the instructions 1008 of FIG. 10. With reference to FIG. 16, the graphical interface 1710 further includes the lanes 1622 and 1632 and the sliders 1624 and 1634, in relation to the axes 1620 and 1630, as well as the rendering 1610 depicting a spatial relationship between the axis or heading of the scope (0,0) and a range of suitable positions for the needle represented by the lanes 1622 and 1632. In the example of FIG. 17B, the graphical interface 1710 is shown to include multiple alignment indications (such as the needle alignment feature 806, the lanes 1622 and 1632, and the sliders 1624 and 1632). However, in actual implementations, the graphical interface 1710 may include only a subset of the alignment indications depicted in FIG. 17B.

As shown in FIG. 17B, the slider 1624 is within the vertical lane 1622 (at a distance of 2 mm away from the scope's axis of heading), which indicates that the position of the needle is aligned with the position and orientation of the scope (at least to a threshold degree) in the AP plane. Similarly, the slider 1634 is within the horizontal lane 1632, which indicates that the position of the needle is aligned with the position and orientation of the scope (at least to a threshold degree) in the CC plane. Further, the needle alignment feature 806 indicates that the needle and the scope are coaxially aligned (as indicated by the tip of the cone pointing out of the page, in a direction orthogonal to the image plane). Because the position and orientation of the needle is aligned (and coaxial) with the position and orientation of the scope, the user proceed to a subsequent step of the percutaneous access procedure.

In the examples of FIGS. 6-17B, the graphical interfaces are configured to depict an alignment and/or coaxiality of a needle and a scope. However, in some other aspects, a graphical interface may be configured to depict an alignment and/or coaxiality of a needle with respect to a target anatomy (such as a calyx and/or a papilla). For example, an alignment indication system may utilize one or more image processing or computer vision techniques to detect or identify the target anatomy based on image data. Example suitable image processing techniques include 3D pose estimation, depth estimation, segmentation, machine learning, and statistical analysis, among other examples. As used herein, the term “segmentation” refers to various techniques for portioning a digital image into groups of pixels (or “image segments”) based on related characteristics or identifying features. Example segmentation techniques include machine learning models, masking, thresholding, clustering, and edge detection, among other examples. After determining a pose of the target anatomy, the alignment indication system may generate a graphical interface indicating an alignment and/or coaxiality of the needle and the target anatomy using any of the techniques described with reference to FIGS. 6-17B.

In some implementations, an alignment indication system may segment image data received from a camera disposed on or proximate to the distal end of the scope and estimate the pose of the target anatomy based on various characteristics or properties of the image segments, for example, using pose estimation and/or scene reconstruction techniques (such as structure from motion, simultaneous localization and mapping (SLAM), or depth estimation). In some other implementations, an alignment indication system may estimate the pose of the target anatomy based on a 3D image of the anatomy. For example, a CT scanner or cone beam CT (CBCT) scanner (also referred to as a “fluoroscope”) may be used to acquire tomographic images (also referred to as “tomograms”) of the anatomy before and/or during the percutaneous access procedure. A tomogram is a cross-section or slice of a 3D volume. For example, multiple tomograms can be stacked or combined to recreate the 3D volume (such as a 3D model of the patient's kidney). Thus, tomograms can be used to detect a precise position and/or orientation (in a 3D image space) of the target anatomy. The alignment indication system may further convert the pose of the target anatomy from the 3D image space to the sensor space based on a transformation matrix that registers the 3D image space to the sensor space.

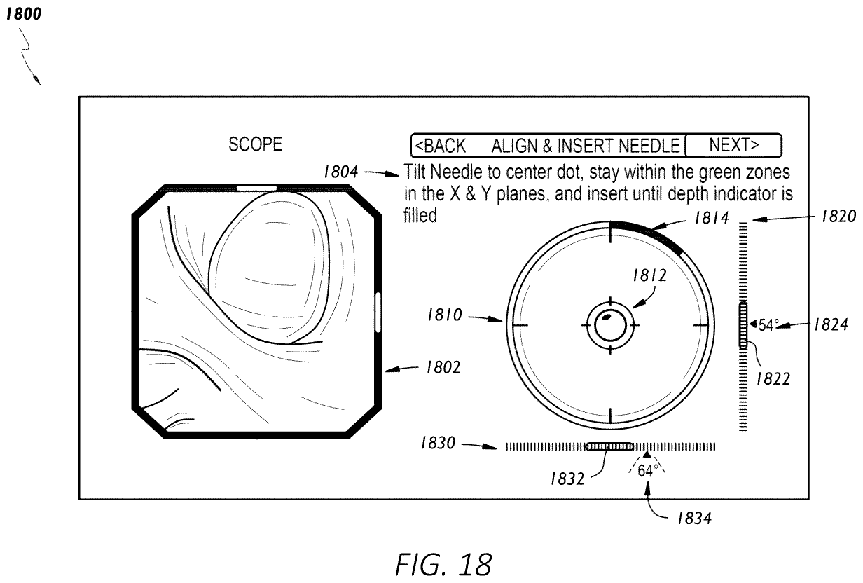

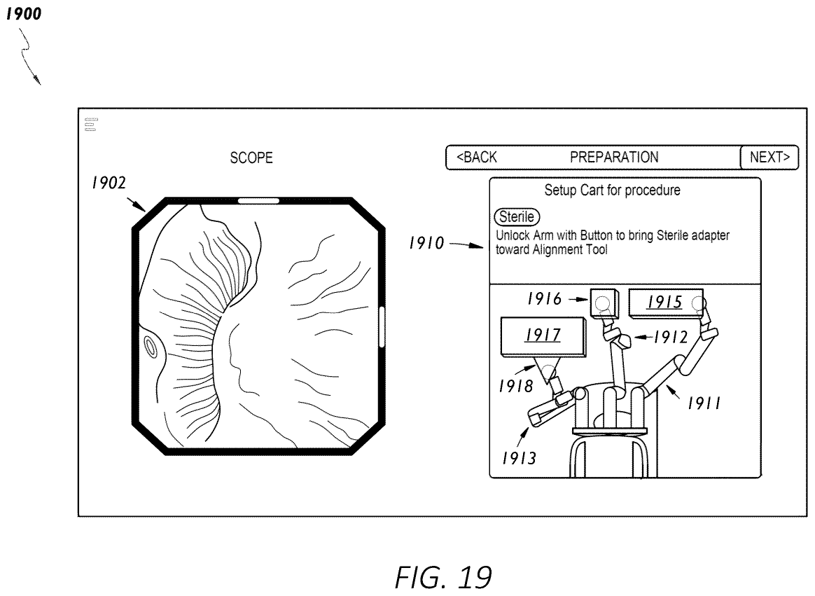

FIG. 18 shows an example graphical interface 1800 providing instrument alignment guidance for needle insertion, according to some implementations. For example, the graphical interface 1800 may be displayed to a user of a medical system (such as on the display 142 of FIG. 1) during a needle insertion phase of a percutaneous access procedure. In some implementations, the graphical interface 1800 may be one example of the instrument-alignment interface 410 of FIG. 3B or the graphical interface 1405 of FIG. 14. More specifically, the graphical interface 1800 may help guide the user to maintain alignment between a needle and a scope while inserting the needle towards a designated target within an anatomy.

The graphical interface 1800 is shown to include an image of the anatomy 1802 and an alignment-progress visualization 1810 that includes an instrument alignment element 1812 and a progress bar 1814. The image of the anatomy 1802 may depict an FOV of a camera disposed on or proximate to the distal end of the scope (such as the image 802 of FIGS. 8-12 and 15). In some implementations, the alignment-progress visualization 1810 may be one example of the alignment-progress visualization 504 of FIG. 3C. For example, the instrument alignment element 1812 may indicate an orientation of the needle relative to the designated target. More specifically, the trajectory of the needle may be aligned with the target when the dot or bubble is centered inside the white inner ring or circle of the alignment-progress visualization 1810. The progress bar 1814 may indicate an insertion depth of the needle or a proximity of the needle to the designated target. For example, the gray outer ring or circumference of the alignment-progress visualization 1810 may “fill” with a different color as the needle is inserted closer towards the designated target.

The graphical interface 1800 further depicts an AP axis 1820 and a CC axis 1830 having “lanes” 1822 and 1832, respectively, overlaid thereon. In some implementations, the lanes 1822 and 1832 may be examples of the alignment ranges 1402 and 1403, respectively, of FIG. 14. More specifically, the height of the vertical lane 1822 depicts a range of suitable positions and/or orientations for the needle to maintain a threshold degree of alignment with the current position and orientation of the scope in the AP plane. Similarly, the width of the horizontal lane 1832 depicts a range of suitable positions and/or orientations for the needle to maintain a threshold degree of alignment with the current position and orientation of the scope in the CC plane. The graphical interface 1800 also includes sliders 1824 and 1834 indicating the current position and/or orientation of the needle with respect to the axes 1820 and 1830, respectively. In some implementations, the graphical interface 1800 may include instructions 1804 to tilt the needle to center the dot within the alignment-progress visualization 1810, while maintaining the sliders 1824 and 1834 within the lanes 1822 and 1832, until the progress bar 1814 is filled.

As shown in FIG. 18, the slider 1824 is within the vertical lane 1822 (at a 54° angle of heading), which indicates that the positions and/or orientations of the needle and the scope are aligned (at least to a threshold degree) in the AP plane. By contrast, the slider 1834 is outside the horizontal lane 1832 (at a 64° angle of heading), which indicates that the positions and/or orientations of the needle and the scope are not aligned in the CC plane. In some implementations, the slider 1834 may include additional coloring and/or graphics to further indicate that the needle and the scope are not aligned in the CC plane. Aspects of the present disclosure recognize that a user may not pay attention to the alignment of the sliders 1824 and 1834 with the lanes 1822 and 1832, respectively, while attempting to orient the trajectory of the needle to rendezvous with the designated target. Thus, in some implementations, the graphical interface 1800 may change the color of the bubble associated with the instrument alignment element 1812 to indicate whether the needle and the scope are coaxially aligned. For example, the bubble may turn red when the needle and the scope are not coaxially aligned (such as shown in FIG. 18).

Aspects of the present disclosure recognize that a graphical interface (such as the interface 144 of FIGS. 1 and 4 or the graphical interface 609 of FIG. 6) can also be used to guide or facilitate setup of the robotic system 110. With reference for example to FIGS. 1-4, a user positions the robotic arms 112 of the robotic system 110 at desired locations proximate to the patient 130 to perform a medical procedure. However, the robotic arms 112 may be physically and/or mechanically limited in how they can be positioned or moved when setting up the robotic system 110. In addition to physical or mechanical limitations, various other factors may further limit the area or volume in which the robotic arms 112 can be positioned during setup. Example limiting factors include the shape and/or dimensions of the medical instrument, the shape and/or physiological characteristics of the patient's luminal network, the location of a target within the anatomy, the working area of the robotic arms, or the type of procedure to be performed, among other examples. Thus, setting up the robotic arms 112 can be a challenging task, particularly when the user does not understand the physical or mechanical constraints of the arms 112. In some aspects, a graphical interface may reduce the cognitive load on a user by displaying real-time guidance indicating a range of achievable movements of one or more robotic arms.

FIG. 19 shows an example graphical interface 1900 for guided positioning of one or more robotic arms, according to some implementations. In some implementations, the graphical interface 1900 may be one example of the graphical interface 609 of FIG. 6 or the interface 144 of FIGS. 1 and 4. For example, the graphical interface 1900 may be generated by the system 600 (or the interface generation component 640) of FIG. 6 and/or by the control system 140 of FIGS. 1-4.

The graphical interface 1900 is shown to include an image 1902 depicting an FOV of a camera disposed on or proximate to the distal end of a scope (such as the scope 120 of FIGS. 1-4). The graphical interface 1900 also includes a visual guide 1910 for positioning the robotic arms 1911-1913. The visual guide 1910 is configured to display real-time information about the current poses of the robotic arms 1911-1913. As described with reference to FIGS. 1-4, the control system 140 can determine the poses (including positions and/or orientations) of the robotic arms 1911-1913 based on user input data, robotic command data, kinematic data, and/or various images or other sensor data captured by the medical system. In some implementations, the robotic arms 1911-1913 may be examples of the robotic arms 112 of FIGS. 1-4.

In the example of FIG. 19, the robotic arms 1911 and 1912 are currently positioned at desired locations proximate to a patient (not shown for simplicity). For example, the robotic arms 1911 and 1912 may be hold and/or manipulate the scope. Thus, the graphical interface 1900 can be displayed to a user of a medical system (such as the medical system 100 or 400 of FIGS. 1 and 4, respectively) while setting up the third robotic arm 1913 for the current procedure. In some implementations, the third robotic arm 1913 may be configured to insert a catheter (such as the catheter 430 of FIG. 4) into a percutaneous access sheath at least partially inserted in the patient. However, in some other implementations, the robotic arm 1913 may be configured to hold and/or manipulate various other medical tools or instruments (such as the EM field generator 180 of FIG. 1).

The visual guide 1910 also displays a visual indication about the range of movement currently achievable by each of the robotic arms 1911-1913. For example, the visual guide 1910 shows that the end effector of the first robotic arm 1911 can move anywhere within a rectangular volume 1915 based on the current position and orientation of the arm 1911. The visual guide 1910 also shows that the end effector of the second robotic arm 1912 can move within a smaller rectangular volume 1916 (compared to the rectangular volume 1915) based on the current position and orientation of the arm 1912. The control system 140 can determine the volumes 1915-1918 or ranges of movement for the robotic arms 1911-1913 based on the current pose of each robotic arm and known mechanical properties and/or limitations of the robotic arm.

In the example of FIG. 19, the visual guide 1910 shows that the end effector of the third robotic arm 1913 can reach any position within a rectangular volume 1917 based on the current pose of the arm 1913. However, the end effector can only enter the rectangular volume 1917 via a cone-shaped path 1918. In other words, the movements of the end effector are confined to the cone 1918 until the end effector reaches the rectangular volume 1917 (at which point the end effector may have full range of movement within the rectangular volume 1917, depending on the position and orientation of the robotic arm 1913 at that time). Because the visual guide 1910 provides real-time information about the achievable ranges of movement, the shapes and/or sizes of the volumes 1915-1917 may vary in response to changes to the positions and/or orientations of the robotic arms 1911-1913. For example, depending on how the user moves or positions the third robotic arm 1913, the size and/or shape of the rectangular volume 1917 may change by the time the end effector reaches the volume 1917.

In some implementations, the control system 140 may signal that one or more of the robotic arms 1911-1913 are at the edges or limits of the volumes 1915-1918 in the form of visual, audible, and/or tactile feedback (such as by changing the color of one or more of the volumes 1915-1918, playing a beeping sound, and/or activating haptics on an input device, among other examples). In some other implementations, the volumes 1915-1918 may be displayed as an overlay on the robotic arms 1911-1913 in an augmented reality (AR) or virtual reality (VR) environment (such as where the user is wearing AR or VR glasses). Still further, in some implementations, the visual guide 1910 may display multiple views (such as from the top, side, and/or front) of the robotic arms 1911-1913 so that the user can assess the volumes 1915-1918 at different angles in 3D space.

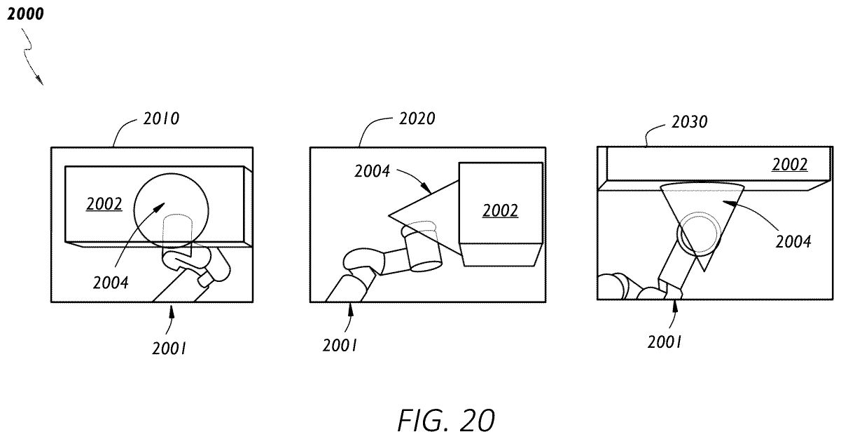

FIG. 20 shows an example visual guide 2000 for positioning a robotic arm 2001, according to some implementations. In some implementations, the visual guide 2000 may be displayed on the graphical interface 1900 of FIG. 19 in addition to, or in lieu of, the visual guide 1910. With reference to FIG. 19, the robotic arm 2001 may be one example of any of the robotic arms 1911-1913. In the example of FIG. 20, the visual guide 2000 includes front, side, and overhead views 2010-2030, respectively, of the robotic arm 2001. Each of the views 2010-2030 shows a rectangular volume 2002 and a cone-shaped volume 2004 in relation to the end effector of the robotic arm 2001. As shown in FIG. 19, the volumes 2002 and 2004 represent achievable ranges (or limits) of movement by the end effector given the current position and orientation of the robotic arm 2001. By displaying the volumes 2002 and 2004 in three different views 2010-2030, the visual guide 2000 may provide the user with better spatial understanding of how the robotic arm 2001 can move in 3D space.

In addition to defining a range of achievable movement for its end effector, the pose of a robotic arm also may limit how far a sterile adapter (coupled to the end effector) can be rotated to align with a percutaneous access sheath. A sterile adapter functions as an interface between a medical instrument (such as a catheter) coupled to the end effector and a sheath that has been percutaneously inserted in a patient. More specifically, the sterile adapter is configured to bring the instrument into alignment with the sheath. To ensure that the instrument is precisely inserted into the percutaneous access sheath, the user may need to rotate the sterile adapter to be properly aligned with an orientation of the sheath. Over-rotating the sterile adapter can apply excess torque on the IDM to which the sterile adapter is attached, which in turn can trigger a system fault. Aspects of the present disclosure recognize that the angular motion limits of the IDM depend on the position and orientation of the robotic arm. Thus, in some aspects, a graphical interface may further provide real-time guidance for aligning a sterile adapter with a percutaneous access sheath based on the pose of a robotic arm.

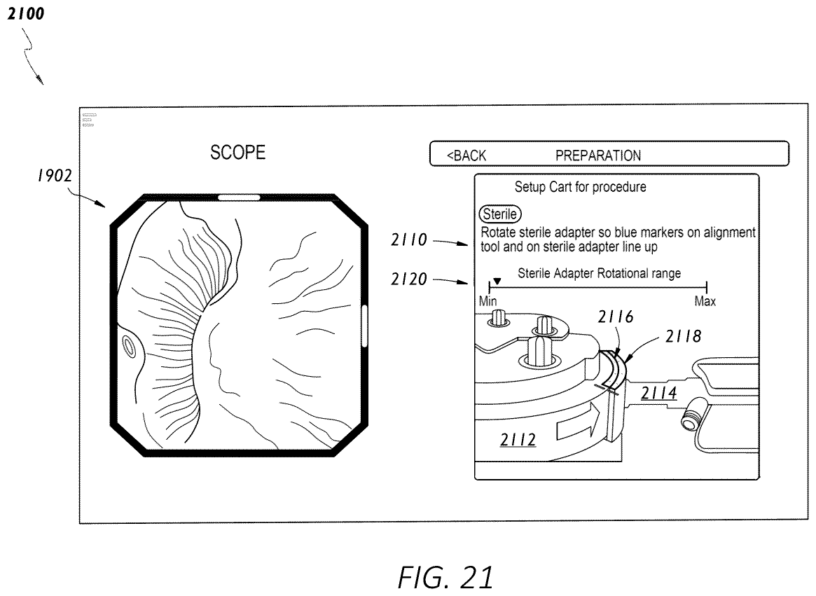

FIG. 21 shows an example graphical interface 2100 for guided alignment of a sterile adapter 2112, according to some implementations. In some implementations, the graphical interface 2100 may be one example of the graphical interface 609 of FIG. 6 or the interface 144 of FIGS. 1 and 4. For example, the graphical interface 2100 may be generated by the system 600 (or the interface generation component 640) of FIG. 6 and/or by the control system 140 of FIGS. 1-4. More specifically, the graphical interface 2100 can be displayed to a user of a medical system (such as the medical system 100 or 400 of FIGS. 1 and 4, respectively) while attempting to align the sterile adapter 2112 with a percutaneous access sheath 2114.

The graphical interface 2100 is shown to include the image 1902 depicting the FOV of the scope of FIG. 19. The graphical interface 2100 also includes a visual guide 2110 for aligning the sterile adapter 2112 with the sheath 2114. More specifically, the visual guide 2110 shows a side view of the sterile adapter 2112 and the sheath 2114. The visual guide 2110 also displays alignment indicators 2116 and 2118 on the sterile adapter 2112 and the sheath 2114, respectively, to provide real-time information about the relative orientations of the instruments as well as a visual indication 2120 about a range of rotation currently achievable by the sterile adapter 2112. The sterile adapter 2112 is properly aligned with the sheath 2114 when the alignment indicator 2116 on the sterile adapter 2112 is aligned with the alignment indicator 2118 on the sheath 2114. Thus, a user may need to rotate the sterile adapter 2112 until the alignment indicators 2116 and 2118 are aligned.

As described above, the allowable range of rotation for the sterile adapter 2112 may depend on the current position and orientation of the robotic arm to which it is coupled (such as how the user moved the robotic arm to the current position). The visual indication 2120 includes a slider (in the shape of a triangle) indicating how much further the sterile adapter 2112 can rotate in a clockwise or counterclockwise direction before reaching the angular motion limits of the underlying IDM (labeled “max” on either side of the slider bar). As shown in FIG. 21, the sterile adapter 2112 is currently near the limit of rotation in a counterclockwise (or clockwise) direction. However, because the alignment indicators 2116 and 2118 are already properly aligned, the user may proceed to insert the catheter (or other medical instrument) into the sterile adapter 2112 in its current configuration. If the alignment indicators 2116 and 2118 could not be aligned without rotating the sterile adapter 2112 beyond the angular motion limits of the underlying IDM, the user may need to reposition the robotic arm to adjust the range of rotation.

In some implementations, the control system 140 may signal that the sterile adapter 2112 is at the edges or limits of the allowable range of rotation in the form of visual, audible, and/or tactile feedback (such as by changing the colors of sterile adapter 2112 and/or the visual indication 2120, playing a beeping sound, and/or activating haptics on an input device). In some other implementations, the visual indication 2120 may be displayed as an overlay on the sterile adapter 2112 in an AR or VR environment (such as where the user is wearing AR or VR glasses). Still further, in some implementations, the visual guide 2110 may display a different view of the sterile adapter 2112 and the sheath 2114 (such as an overhead view) so that the user can assess the allowable range of rotation from a different angle.

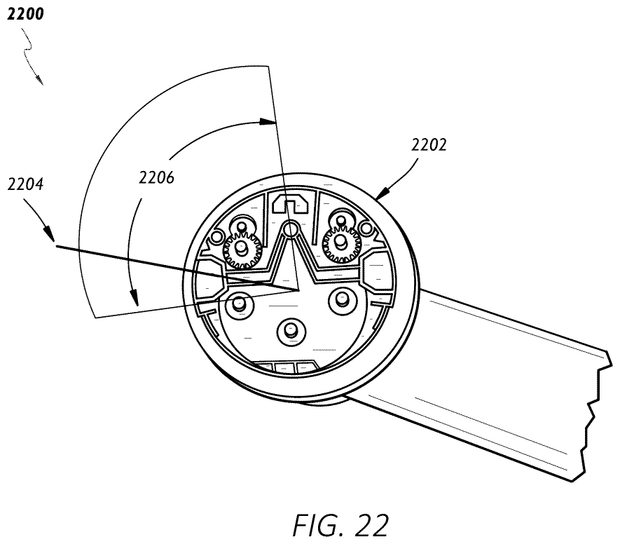

FIG. 22 shows an example visual guide 2200 for aligning a sterile adapter 2202 with a percutaneous access sheath (not shown for simplicity), according to some implementations. In some implementations, the visual guide 2200 may be displayed on the graphical interface 2100 of FIG. 21 in addition to, or in lieu of, the visual guide 2110. With reference to FIG. 21, the sterile adapter 2202 may be one example of the sterile adapter 2112. In the example of FIG. 22, the visual guide 2200 shows an overhead (or top-down) view of the sterile adapter 2202. The visual guide 2200 also shows a range of rotation 2206 currently available for rotating the sterile adapter 2202 (depicted as a shaded region overlapping the upper left quadrant of the sterile adapter 2202) as well as an indication 2204 of how far the sterile adapter 2202 is already rotated within the range 2206 (denoted as a line pointing radially outward from the center of the sterile adapter 2202). With reference for example to FIG. 21, the indication 2204 may represent the triangular-shaped slider feature, and the range of rotation 2206 may represent the underlying slider bar, of the visual indication 2120.

Aspects of the present disclosure further recognize that a graphical interface (such as the interface 144 of FIGS. 1 and 4 or the graphical interface 609 of FIG. 6) can be used to dynamically display instrument controls (such as one or more control schemes for an input device). With reference for example to FIGS. 1-4, a user may provide inputs to the robotic system 110 via the input device 146 associated with the control system 140 to navigate an instrument (such as a scope or a catheter) within an anatomy and/or to perform various functions provided by the instrument (such as lasing a stone into smaller fragments or opening and closing a basket around a stone). Because different instruments may support different features and/or functions, the control system 140 may map (and remap) various buttons and/or joysticks of the input device 146 with different controls depending on the instrument currently in use. The myriad controls may be challenging for a user to remember, particularly when switching between different instruments. In some aspects, a graphical interface may reduce the cognitive load on a user by displaying contextual controls for an input device. As used herein, the term “contextual controls” refers to any control scheme (such as a mapping of instrument controls to various user inputs) that is specific to a particular context and/or instrument being used.

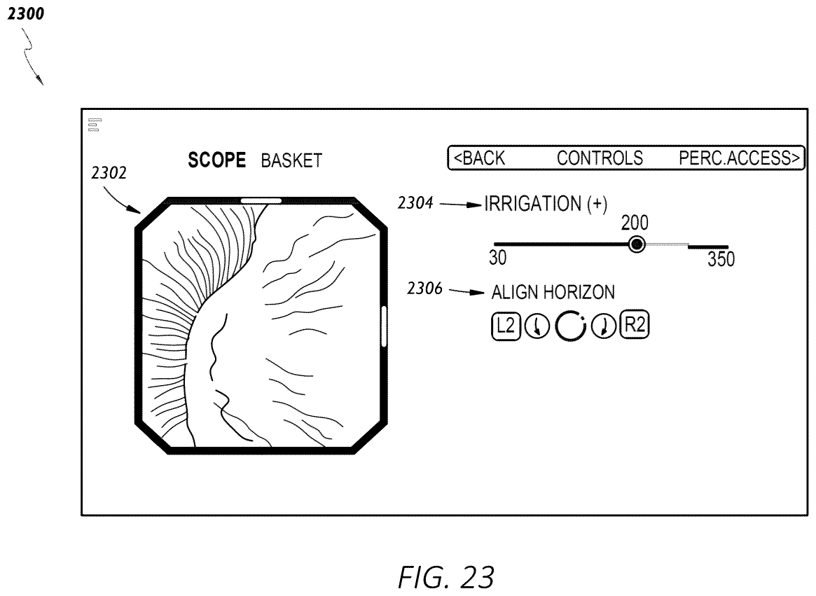

FIG. 23 shows an example graphical interface 2300 for controlling a scope, according to some implementations. In some implementations, the graphical interface 2300 may be one example of the graphical interface 609 of FIG. 6 or the interface 144 of FIGS. 1 and 4. For example, the graphical interface 2300 may be generated by the system 600 (or the interface generation component 640) of FIG. 6 and/or by the control system 140 of FIGS. 1-4. More specifically, the graphical interface 2300 may be displayed to a user of a medical system (such as the medical system 100 or 400 of FIGS. 1 and 4, respectively) while the user is navigating a scope within an anatomy (such as the scope 120 of FIGS. 1 and 4). In the example of FIG. 23, the control system 140 can detect that the user is currently controlling the scope based on user input associated with instrument selection (such as described with reference to FIG. 4).

The graphical interface 2300 is shown to include an image 2302 depicting an FOV of a camera disposed on or proximate to the distal end of the scope. The graphical interface 2300 also includes an irrigation control feature 2304 and an “align horizon” feature 2306. The align horizon feature 2306 rotates the scope to reorient its primary plane and its secondary plane. For example, the scope may have greater reach or articulation in its primary plane compared to its secondary plane. Thus, if a user is unable to achieve a desired degree of articulation with the current orientation of the instrument, the user may use the align horizon feature 2306 to rotate the scope so that the primary plane switches with the secondary plane. In some implementations, the align horizon feature 2306 may be activated via an input device coupled to a robotic system that controls the movement of the scope (such as the controller 500 of FIGS. 5A and 5B). As shown in FIG. 23, the user may activate the align horizon feature 2306 by pressing the “L2” and “R2” buttons 528 and 524, respectively, on the controller 500.

The irrigation control feature 2304 includes a circular slider showing the current pressure and/or flow rate (200) of irrigation fluid, as well as a maximum (350) and minimum (30) flow rate and/or pressure. Irrigation can be used to achieve distention of the anatomy (such as for endoscopic vision), maintain suitable intrarenal pressures during a medical procedure (such as to prevent damage to the anatomy), or move objects (such as kidney stones) around within the anatomy. Improper management of irrigation (and aspiration) can adversely affect the health of the patient and/or efficacy of the procedure. For example, over-pressurization of the anatomy can result in fractures, tissue breakage, or damage to the anatomy. On the other hand, under-pressurization can result in insufficient anatomical distention that is otherwise needed for visualization. The irrigation control feature 2304 allows the user to control and monitor the flow rate and/or pressure of irrigation while navigating the scope. In some implementations, a user may interact with the irrigation control feature 2304 by dragging the slider on the graphical interface 2300. In some other implementations, a user may move the slider of the irrigation control feature 2304 using another input device (such as the controller 500 of FIGS. 5A and 5B).

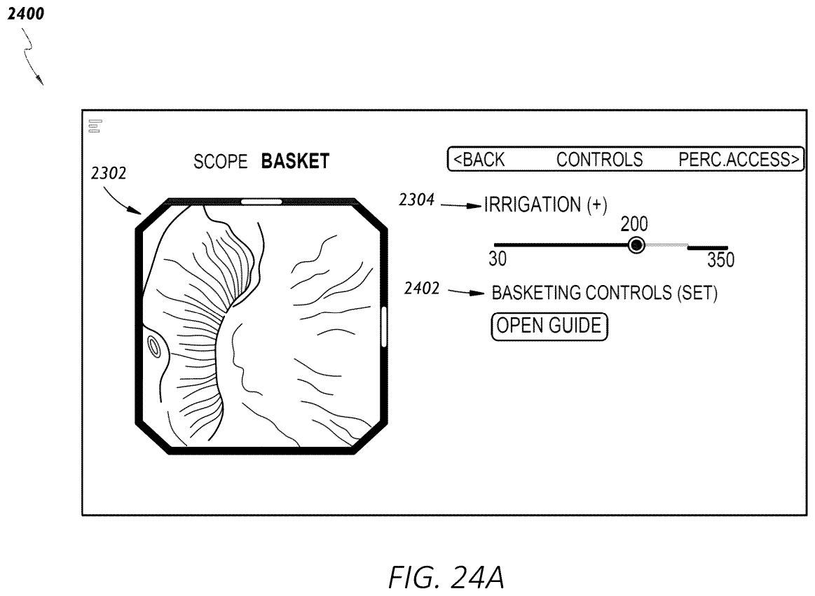

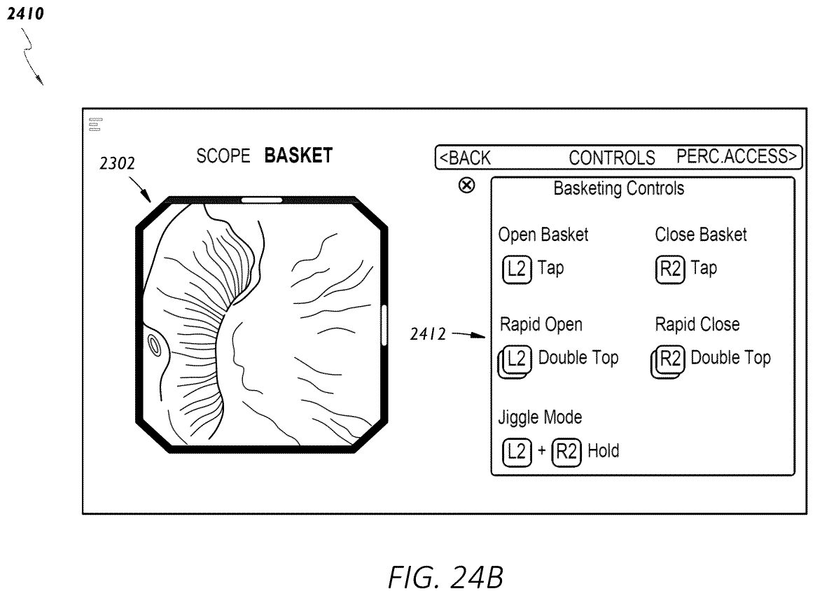

FIG. 24A shows an example graphical interface 2400 for controlling a basket retrieval device, according to some implementations. In some implementations, the graphical interface 2400 may be one example of the graphical interface 609 of FIG. 6 or the interface 144 of FIGS. 1 and 4. For example, the graphical interface 2400 may be generated by the system 600 (or the interface generation component 640) of FIG. 6 and/or by the control system 140 of FIGS. 1-4. More specifically, the graphical interface 2400 may be displayed to a user of a medical system (such as the medical system 100 or 400 of FIGS. 1 and 4, respectively) while the user is controlling a basket retrieval device to capture, move, or break up kidney stones within an anatomy. In the example of FIGS. 24A and 24B, the control system 140 can detect that the user is currently controlling the basket retrieval device based on user input associated with instrument selection (such as described with reference to FIG. 4).

The graphical interface 2400 is shown to include the image 2302 depicting the FOV of the scope and the irrigation control feature 2304. The graphical interface 2400 also includes a basketing controls feature 2402 that can be activated to display a guide indicating various input controls for operating the basket retrieval device. In some implementations, a user may interact with the basketing controls feature 2402 by tapping or clicking the “open guide” icon on the graphical interface 2400. In some other implementations, the basketing controls feature 2402 may be activated via an input device coupled to a robotic system that controls the operation of the basket retrieval device (such as the controller 500 of FIGS. 5A and 5B). As shown in FIG. 24A, the user may activate the basketing controls feature 2402 by pressing a “set” button on the controller 500 (which may be one of the additional buttons 512) or on another input device.

FIG. 24B shows another example graphical interface 2410 for controlling a basket retrieval device, according to some implementations. In some implementations, the graphical interface 2410 may be one example of the graphical interface 609 of FIG. 6 or the interface 144 of FIGS. 1 and 4. For example, the graphical interface 2410 may be generated by the system 600 (or the interface generation component 640) of FIG. 6 and/or by the control system 140 of FIGS. 1-4. More specifically, the graphical interface 2410 may be displayed after a user activates the basketing controls feature 2402 on the graphical interface 2400 of FIG. 24A.

The graphical interface 2410 is shown to include the image 2302 depicting the FOV of the scope. The graphical interface 2410 also includes a basketing controls modal 2412 that displays a mapping of various user inputs to various functions of the basket retrieval device. In some other implementations, the basket retrieval device may be controlled via an input device (such as the controller 500 of FIGS. 5A and 5B). As shown in FIG. 24B, the user may open the basket by tapping the “L2” button 528 on the controller 500 and may open the basket more quickly by double tapping the “L2” button 528. By contrast, the user may close the basket by tapping the “R2” button 524 on the controller 500 and may close the basket more quickly by double tapping the “R2” button 524. The user also may jiggle the basket by concurrently pressing and holding the “L2” and “R2” buttons 528 and 524, respectively, of the controller 500.

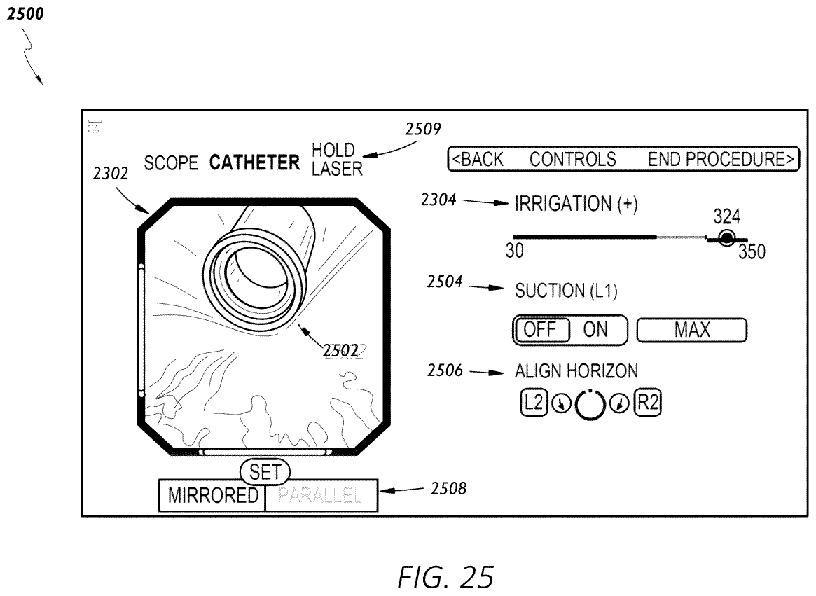

FIG. 25 shows an example graphical interface 2500 for controlling a catheter, according to some implementations. In some implementations, the graphical interface 2500 may be one example of the graphical interface 609 of FIG. 6 or the interface 144 of FIGS. 1 and 4. For example, the graphical interface 2500 may be generated by the system 600 (or the interface generation component 640) of FIG. 6 and/or by the control system 140 of FIGS. 1-4. More specifically, the graphical interface 2500 may be displayed to a user of a medical system (such as the medical system 100 of FIG. 1) while the user is navigating a catheter 2502 within an anatomy. In the example of FIG. 25, the control system 140 can detect that the user is currently controlling the catheter 2502 based on user input associated with instrument selection (such as described with reference to FIG. 4). In some implementations, the catheter 2502 may be one example of the catheter 430 of FIG. 4.

The graphical interface 2500 is shown to include the image 2302 depicting the FOV of the scope and the irrigation control feature 2304. In the example of FIG. 25, the image 2302 shows a third-person perspective of the catheter 2502 in relation to the scope and the surrounding anatomy. The graphical interface 2500 also includes a suction control feature 2504, an align horizon feature 2506, and a drive mode feature 2508. The drive mode feature 2508 indicates the current drive mode of the catheter 2502 (such as the mirrored mode or the parallel mode described with reference to FIG. 4). The drive mode feature 2508 also indicates a user input for switching between the drive modes. In some implementations, the user may switch the drive mode via an input device coupled to a robotic system that controls the movement of the catheter 2502 (such as the controller 500 of FIGS. 5A and 5B). As shown in FIG. 25, the user may switch between the mirrored mode and the parallel mode by pressing a “set” button on the controller 500 (which may be one of the additional buttons 512) or on another input device.

The align horizon feature 2506 rotates the catheter 2502 to reorient its primary plane and its second plane. For example, the catheter 2502 may have greater reach or articulation in its primary plane compared to its secondary plane (similar to the scope). Thus, the align horizon feature 2506 may rotate the scope so that the primary plane switches with the secondary plane (similar to the align horizon feature 2306 of FIG. 23). In some implementations, the align horizon feature 2506 may be activated via an input device coupled to a robotic system that controls the movement of the catheter 2502 (such as the controller 500 of FIGS. 5A and 5B). As shown in FIG. 25, the user may activate the align horizon feature 2506 by pressing the “L2” and “R2” buttons 528 and 524, respectively, on the controller 500.

The suction control feature 2504 controls an aspiration function of the catheter 2502. As described with reference to FIG. 23, improper management of irrigation and aspiration can adversely affect the health of the patient and/or efficacy of a medical procedure. The suction control feature 2504 allows the user to control and monitor aspiration via the catheter 2502 while navigating the catheter 2502. In some implementations, a user may interact with the suction control feature 2504 by tapping or clicking the “off/on” or “max” icons on the graphical interface 2500. For example, where the graphical interface 2500 is displayed on a touchscreen display, tapping on the “off/on” icon may toggle the aspiration off and on, whereas tapping on the “max” icon may activate a maximum amount of suction pressure. In some other implementations, a user may toggle the “off/on” or “max” icons of the suction control feature 2504 using a separate input device (such as the controller 500 of FIGS. 5A and 5B).

In some implementations, the graphical interface 2500 may further include an indication 2509 for how to control a laser (such as for lithotripsy). In some implementations, a user may toggle between the controls for various instruments (such as the scope and the catheter) by tapping or pressing a button (such as the “L1” button 526) on the controller 500. However, tapping the same button to toggle the controls for the laser may cause some users to inadvertently insert or retract the laser while attempting to drive the scope or the catheter. Thus, in some implementations, a user may be required to tap and hold a button to engage the laser controls (such as shown by the indication 2509). In other words, the user may control an insertion or retraction of the laser only while holding the assigned button. This ensures that any movement of the laser is an intentional act. In some implementations, the user may control the laser by holding the “R1” button 522 while inserting or retracting the laser using the left joystick 514.

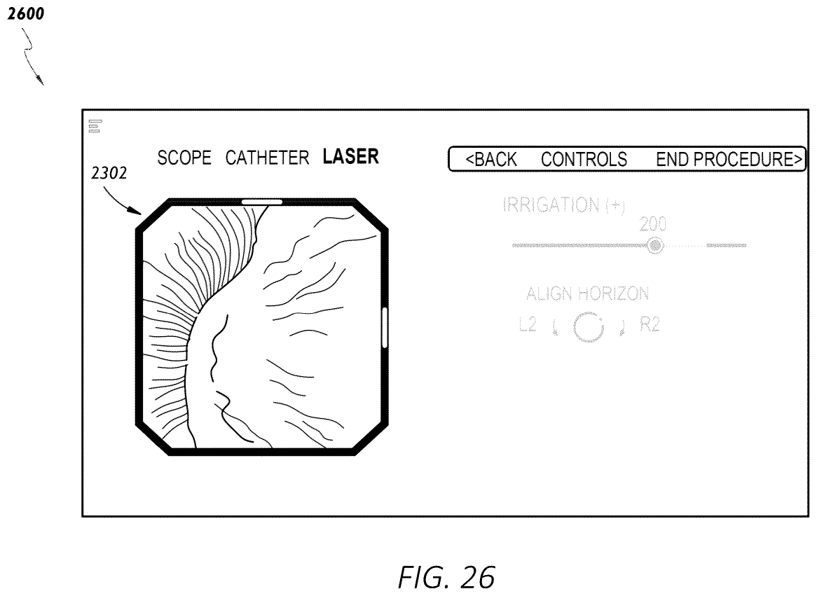

FIG. 26 shows an example graphical interface 2600 for controlling a laser, according to some implementations. In some implementations, the graphical interface 2600 may be one example of the graphical interface 609 of FIG. 6 or the interface 144 of FIGS. 1 and 4. For example, the graphical interface 2600 may be generated by the system 600 (or the interface generation component 640) of FIG. 6 and/or by the control system 140 of FIGS. 1-4. More specifically, the graphical interface 2600 may be displayed to a user of a medical system (such as the medical system 100 or 400 of FIGS. 1 and 4) only while the user is holding an assigned button on an input device (such as described with reference to FIG. 25).

The graphical interface 2600 is shown to include the image 2302 depicting the FOV of the scope. However, the irrigation control feature and the align horizon feature on the right side of the graphical interface 2600 are grayed out (or hidden) to provide a visual indication that the user is currently in control of the laser (and not the scope or the catheter). While holding down the assigned button, the user may cause the laser to insert and/or retract using another input component or device (such as by tilting the joystick 514 of the controller 500 up or down). However, once the user releases the assigned button, the graphical interface 2600 may revert to a graphical interface for controlling the scope or the catheter (such as any of the graphical interfaces 2300 or 2500 of FIGS. 23 and 25, respectively).

Any specific text, fonts, shapes, buttons, icons, or other graphical features shown in any of the graphical interfaces depicted in FIGS. 8-12, 15-19, 21, and 23-26 are intended to be illustrative rather than restrictive. These examples are provided to demonstrate the principles of the present disclosure and to highlight various types of information and/or system controls that can be displayed on a graphical interface. Various modifications, substitutions, alterations, and adaptations can be made to the examples herein without departing from the scope of the present disclosure. In some aspects, the graphical interfaces also may be customized to user preferences. Example suitable customization options may include, among other examples, changing the sizes or locations of images, changing the sizes or locations of the renderings, changing the colors of the scope and/or needle alignment features, changing the colors of the lanes and/or sliders, adjusting various rendering parameters (such as color, opacity, or intensity of various features), or changing the colors of text or highlights in any of the graphical interfaces. Specific features, structures, or characteristics described in connection with any particular example are included for illustration and clarity of understanding only and should not be interpreted as limiting the claims.

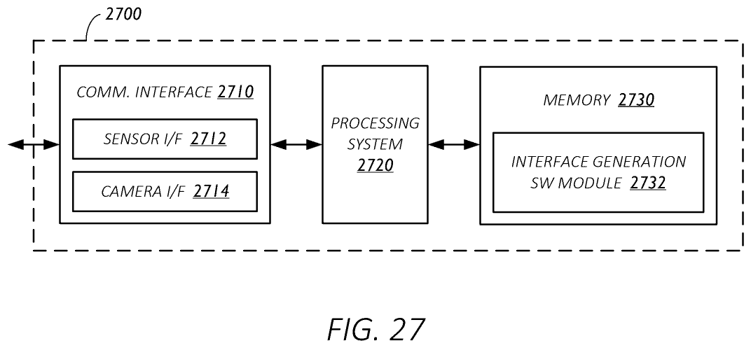

FIG. 27 shows a block diagram of an example control system 2700 for guiding percutaneous access, according to some implementations. In some implementations, the control system 2700 may be one example of the system 600 of FIG. 6 or the control system 140 of FIGS. 1-4. More specifically, the control system 2700 is configured to provide guidance for setting up and/or performing a percutaneous access procedure.

The controller 2700 includes a communication interface 2710, a processing system 2720, and a memory 2730. The communication interface 2710 is configured to communicate with one or more components of the medical system. More specifically, the communication interface 2710 includes a sensor interface (I/F) 2712 for communicating with one or more sensors (such as an EM sensor) and a camera interface (I/F) 2714 for communicating with one or more image sources (such as a camera).

In some implementations, the sensor interface 2712 may receive first sensor data via a sensor disposed on a first instrument within an anatomy and may receive second sensor data via a sensor disposed on a second instrument external to the anatomy, where the first sensor data indicates a pose of the first instrument and the second sensor data indicates a pose of the second instrument. In some implementations, the camera interface 2714 may receive an image depicting a portion of the anatomy in an FOV of a camera disposed on or proximate to a distal end of the first instrument.

The memory 2730 may include a non-transitory computer-readable medium (including one or more nonvolatile memory elements, such as EPROM, EEPROM, Flash memory, or a hard drive, among other examples) that may store an interface generation software (SW) module 2732 to generate a graphical interface that includes the image and an instrument alignment feature indicating an alignment of the second instrument with the FOV of the camera based at least in part on the first sensor data and the second sensor data.

The processing system 2720 includes any suitable one or more processors capable of executing scripts or instructions of one or more software programs stored in the controller 2700 (such as in the memory 2730). For example, the processing system 2720 may execute the interface generation SW module 2732 to generate a graphical interface that includes the image and an instrument alignment feature indicating an alignment of the second instrument with the FOV of the camera based at least in part on the first sensor data and the second sensor data.

In some implementations, execution of the interface generation SW module 2732 may further cause the processing system 2720 to determine a pose of a robotic arm configured to manipulate the first instrument or the second instrument, determine a range of movement achievable by the robotic arm based on the pose of the robotic arm, and display, on the graphical interface, a first visual guide depicting the range of movement achievable by the robotic arm.

In some other implementations, execution of the interface generation SW module 2732 may further cause the processing system 2720 to determine whether the first instrument or the second instrument is being controlled by a user and display, on the graphical interface, a first control scheme or a second control scheme based on whether the first instrument or the second instrument is being controlled by the user, where the first control scheme indicates a mapping of user inputs to controls for the first instrument and the second control scheme indicates a mapping of user inputs to controls for the second instrument.

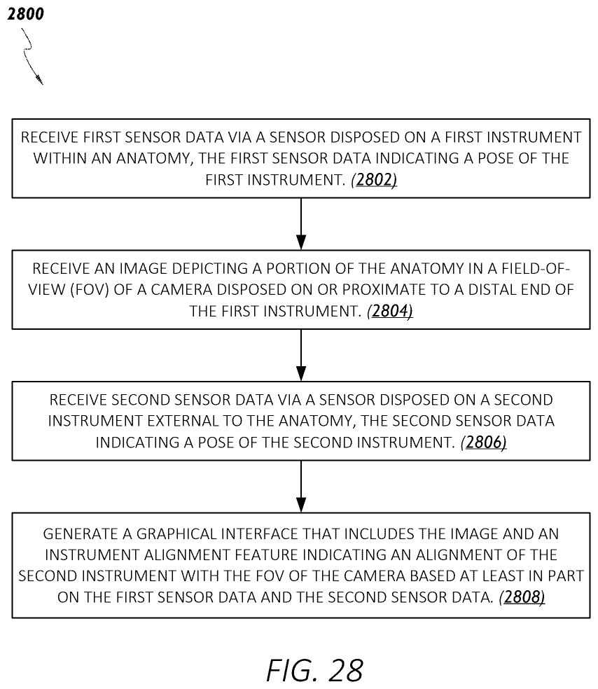

FIG. 28 shows an illustrative flowchart depicting an example operation 2800 for guiding percutaneous access, according to some implementations. In some implementations, the example operation 2800 may be performed by a control system such as the control system 2700 of FIG. 27, the system 600 of FIG. 6, or the control system 140 of FIGS. 1-4.

The control system receives first sensor data via a sensor disposed on a first instrument within an anatomy, the first sensor data indicating a pose of the first instrument (2802). The control system also receives an image depicting a portion of the anatomy in an FOV of a camera disposed on or proximate to a distal end of the first instrument (2804). The control system receives second sensor data via a sensor disposed on a second instrument external to the anatomy, the second sensor data indicating a pose of the second instrument (2806). The control system further generates a graphical interface that includes the image and an instrument alignment feature indicating an alignment of the second instrument with the FOV of the camera based at least in part on the first sensor data and the second sensor data (2808).

In some aspects, the instrument alignment feature may include a 3D model of the second instrument. In some implementations, the 3D model may include a cone having an orientation indicating a coaxiality of the first instrument with the second instrument. In some other implementations, the 3D model may include a rectangular plane intersecting a circle at an orientation indicating a coaxiality of the first instrument with the second instrument. In some implementations, the generating of the graphical interface may include mapping the 3D model to a first coordinate space associated with the first and second sensor data based on the pose of the second instrument, converting the 3D model from the first coordinate space to a second coordinate space associated with the camera based at least in part on the pose of the first instrument, and transforming the 3D model in the second coordinate space to a 2D projection on the image based on one or more intrinsic parameters of the camera.

In some aspects, the control system may further display, on the graphical interface, a coaxiality feature indicating whether the second instrument is coaxial with the first instrument based on the first sensor data and the second sensor data. In some implementations, the coaxiality feature may indicate whether the second instrument is coaxial with the first instrument in an anterior and posterior (AP) plane, a cranial and caudal (CC) plane, or a combination thereof.

In some other aspects, the instrument alignment feature may depict an orientation of each of the first instrument and the second instrument in relation to an AP plane, a CC plane, or a combination thereof. In some implementations, the instrument alignment feature may further indicate a range of suitable orientations for the second instrument based on the orientation of the first instrument, the range of suitable orientations being associated with a threshold degree of coaxiality between the first and the second instruments.

In some aspects, the control system may further determine a pose of a robotic arm configured to manipulate the first instrument or the second instrument, determine a range of movement achievable by the robotic arm based on the pose of the robotic arm, and display, on the graphical interface, a first visual guide depicting the range of movement achievable by the robotic arm. In some implementations, the control system may further detect a change in the pose of the robotic arm and update the first visual guide to depict a new range of movement achievable by the robotic arm based on the change in pose of the robotic arm. In some other implementations, the control system may further determine a range of rotation achievable by a sterile adapter coupled to the robotic arm based on the pose of the robotic arm and display, on the graphical interface, a second visual guide depicting the range of rotation achievable by the sterile adapter.

In some other aspects, the control system may further determine whether the first instrument or the second instrument is being controlled by a user and display, on the graphical interface, a first control scheme or a second control scheme based on whether the first instrument or the second instrument is being controlled by the user, where the first control scheme indicates a mapping of user inputs to controls for the first instrument and the second control scheme indicates a mapping of user inputs to controls for the second instrument. In some implementations, the control system may further determine whether the user is holding an assigned button of an input device and display, on the graphical interface, a third control scheme responsive to determining that the user is holding the assigned button, where the third control scheme indicates a mapping of user inputs to controls for a third instrument. In some implementations, the third instrument may include a laser and the third control scheme may be displayed only while the user is holding the assigned button.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A method for guiding percutaneous access, comprising:
receiving first sensor data via a sensor disposed on a first instrument within an anatomy, the first sensor data indicating a pose of the first instrument;
receiving an image depicting a portion of the anatomy in a field-of-view (FOV) of a camera disposed on or proximate to a distal end of the first instrument;
receiving second sensor data via a sensor disposed on a second instrument external to the anatomy, the second sensor data indicating a pose of the second instrument; and
generating a graphical interface that includes the image and an instrument alignment feature indicating an alignment of the second instrument with the FOV of the camera based at least in part on the first sensor data and the second sensor data.

2. The method of claim 1, wherein the instrument alignment feature includes a three-dimensional (3D) model of the second instrument.

3. The method of claim 2, wherein the 3D model comprises a cone having an orientation indicating a coaxiality of the first instrument with the second instrument.

4. The method of claim 2, wherein the 3D model comprises a rectangular plane intersecting a circle at an orientation indicating a coaxiality of the first instrument with the second instrument.

5. The method of claim 2, wherein the generating of the graphical interface comprises:
mapping the 3D model to a first coordinate space associated with the first and second sensor data based on the pose of the second instrument;
converting the 3D model from the first coordinate space to a second coordinate space associated with the camera based at least in part on the pose of the first instrument; and
transforming the 3D model in the second coordinate space to a two-dimensional (2D) projection on the image based on one or more intrinsic parameters of the camera.

6. The method of claim 1, further comprising:
displaying, on the graphical interface, a coaxiality feature indicating whether the second instrument is coaxial with the first instrument based on the first sensor data and the second sensor data.

7. The method of claim 6, wherein the coaxiality feature indicates whether the second instrument is coaxial with the first instrument in an anterior and posterior (AP) plane, a cranial and caudal (CC) plane, or a combination thereof.

8. The method of claim 1, wherein the instrument alignment feature depicts an orientation of each of the first instrument and the second instrument in relation to an AP plane, a CC plane, or a combination thereof.

9. The method of claim 8, wherein the instrument alignment feature further indicates a range of suitable orientations for the second instrument based on the orientation of the first instrument, the range of suitable orientations being associated with a threshold degree of coaxiality between the first and the second instruments.

10. The method of claim 1, further comprising:
determining a pose of a robotic arm configured to manipulate the first instrument or the second instrument;
determining a range of movement achievable by the robotic arm based on the pose of the robotic arm; and
displaying, on the graphical interface, a first visual guide depicting the range of movement achievable by the robotic arm.

11. The method of claim 10, further comprising:
detecting a change in the pose of the robotic arm; and
updating the first visual guide to depict a new range of movement achievable by the robotic arm based on the change in pose of the robotic arm.

12. The method of claim 10, further comprising:
determining a range of rotation achievable by a sterile adapter coupled to the robotic arm based on the pose of the robotic arm; and
displaying, on the graphical interface, a second visual guide depicting the range of rotation achievable by the sterile adapter.

13. The method of claim 1, further comprising:
determining whether the first instrument or the second instrument is being controlled by a user; and
displaying, on the graphical interface, a first control scheme or a second control scheme based on whether the first instrument or the second instrument is being controlled by the user, the first control scheme indicating a mapping of user inputs to controls for the first instrument and the second control scheme indicating a mapping of user inputs to controls for the second instrument.

14. The method of claim 13, further comprising:
determining whether the user is holding an assigned button of an input device; and
displaying, on the graphical interface, a third control scheme responsive to determining that the user is holding the assigned button, the third control scheme indicating a mapping of user inputs to controls for a third instrument.

15. The method of claim 14, wherein the third instrument comprises a laser and the third control scheme is displayed only while the user is holding the assigned button.

16. A control system for guiding percutaneous access, comprising:
a processing system; and
a memory storing instructions that, when executed by the processing system, cause the control system to:
receive first sensor data via a sensor disposed on a first instrument within an anatomy, the first sensor data indicating a pose of the first instrument;
receive an image depicting a portion of the anatomy in a field-of-view (FOV) of a camera disposed on or proximate to a distal end of the first instrument;
receive second sensor data via a sensor disposed on a second instrument external to the anatomy, the second sensor data indicating a pose of the second instrument; and
generate a graphical interface that includes the image and an instrument alignment feature indicating an alignment of the second instrument with the FOV of the camera based at least in part on the first sensor data and the second sensor data.

17. The control system of claim 16, wherein the instrument alignment feature includes a three-dimensional (3D) model of the second instrument, the generating of the graphical interface comprising:
mapping the 3D model to a first coordinate space associated with the first and second sensor data based on the pose of the second instrument;
converting the 3D model from the first coordinate space to a second coordinate space associated with the camera based at least in part on the pose of the first instrument; and
transforming the 3D model in the second coordinate space to a two-dimensional (2D) projection on the image based on one or more intrinsic parameters of the camera.

18. The control system of claim 16, wherein execution of the instructions further causes the control system to:
display, on the graphical interface, a coaxiality feature indicating whether the second instrument is coaxial with the first instrument based on the first sensor data and the second sensor data.

19. The control system of claim 16, wherein execution of the instructions further causes the control system to:
determine a pose of a robotic arm configured to manipulate the first instrument or the second instrument;
determine a range of movement achievable by the robotic arm based on the pose of the robotic arm; and
display, on the graphical interface, a first visual guide depicting the range of movement achievable by the robotic arm.

20. The control system of claim 16, wherein execution of the instructions further causes the control system to:
determine whether the first instrument or the second instrument is being controlled by a user; and
display, on the graphical interface, a first control scheme or a second control scheme based on whether the first instrument or the second instrument is being controlled by the user, the first control scheme indicating a mapping of user inputs to controls for the first instrument and the second control scheme indicating a mapping of user inputs to controls for the second instrument.
